(19) United States
(12) Patent Application Publication
(10) Pub. No.: US 2013/0324522 A1
(43) Pub. Date: Dec. 5, 2013

(54) INSECT ODORANT RECEPTOR ANTAGONISTS

(71) Applicants: Leslie Vosshall, New York, NY (US); Takao Nakagawa, New York, NY (US)

(72) Inventors: Leslie Vosshall, New York, NY (US); Takao Nakagawa, New York, NY (US)

(73) Assignee: THE ROCKEFELLER UNIVERSITY, New York, NY (US)

(21) Appl. No.: 13/993,940

(22) PCT Filed: Dec. 16, 2011
(86) PCT No.: PCT/US11/65536

§ 371 Date: Aug. 23, 2013

Related U.S. Application Data
(60) Provisional application No. 61/424,455, filed on Dec. 17, 2010.

(51) Int. Cl. A01N 43/56; A01N 43/84; C07D 401/06; C07D 401/10; A01N 43/38; C07D 413/14; C07D 231/12; A01N 43/60; C07D 209/08; C07D 405/12; A01N 43/80

(52) U.S. Cl. CPC A01N 43/56; C07D 405/12; A01N 43/84; C07D 401/06; C07D 401/10; A01N 43/80; C07D 413/14; C07D 231/12; A01N 43/60; C07D 209/08; A01N 43/38
USPC 514/227.8; 514/236.2; 514/307; 514/326; 514/380; 514/235.8; 514/254.07; 514/415

(57) ABSTRACT

Insect repellent compositions are disclosed which are antagonists of insect odorant receptors. These compositions are useful as insect repellents and confusants. These insect repellent compositions may be suitable for topical application. Also disclosed are methods for interfering with the ability of an insect to detect odors. This is accomplished by exposing an insect to an olfactory-disrupting concentration of a compound.

The abstract is shown below without the amendment markings:

Insect repellent compositions are disclosed which are antagonists of insect odorant receptors. These compositions are useful as insect repellents and confusants. These insect repellent compositions may be suitable for topical application. Also disclosed are methods for interfering with the ability of an insect to detect odors. This is accomplished by exposing an insect to an olfactory-disrupting concentration of a compound.

### CROSS REFERENCE TO RELATED APPLICATIONS

This application claims priority from U.S. Provisional Application 61/424,455, filed Dec. 17, 2010, the entire contents of which is incorporated herein by reference.

### FEDERALLY SPONSORED RESEARCH

The following invention was made with Government support under contract number RO1 DC008600 awarded by NIH/NIDCD. The Government has certain rights in this invention.

### FIELD OF THE INVENTION

The invention relates to compounds that are antagonists of odorant receptors of diverse insects. Therefore, these compounds are useful to control the behavior of insect pests that damage crops or spread human and animal infectious diseases. Such compounds may enhance or replace current insect repellents.

### BACKGROUND OF THE INVENTION

Biting insects spread a number of deadly infectious diseases to humans, including malaria, dengue fever, yellow fever, and West Nile encephalitis. Mosquitoes, one of the main insect vectors of these diseases, are attracted to human hosts primarily through their sense of smell. Insect repellents, including those containing DEET (N,N-diethyl-m-toluamide), can be effective in warding off biting insects and thus may reduce both nuisance biting and infectious disease transmission. However, existing insect repellent active ingredients date from the 1950s-1980s and there has been little innovation in this industry aside from reformulating new products with old chemistry. There is a compelling argument to develop alternatives to DEET, which must be reapplied frequently to skin at very high doses to be effective and is not approved for use on young children. Its mechanism of action is controversial and it may act on numerous protein targets in insects as well as showing off-target effects in humans. There is a need for 21^{st} century innovation to design compounds that interfere with the insect sense of smell.

Recent advances in the study of insect olfaction have pinpointed the insect odorant receptors (ORs) as the proteins that detect human odor cues and guide insect host-seeking behavior. Insect ORs are very diverse, with different insects having different receptors, but in all insects a common co-receptor protein (called Or83b in Drosophila flies or OR7 in mosquitoes) assembles with each of the diverse odor-specific subunits to make functional odor-sensitive receptors. Antagonists that target this complex by acting on the common co-receptor may prove to be useful as novel insect repellents or “confusants.” Depending on the population and the desired effect, it may be advantageous to have a broad-spectrum repellent against many different insects that targets the conserved co-receptor or, alternatively, to have a compound specific to one insect species. Such a product could have utility in public health applications, to reduce the biting of insect vectors of disease and in agricultural applications, to reduce damage of food crops by insect pests.

### SUMMARY OF THE INVENTION

It is clear that new treatments are needed for repelling insects. The current invention demonstrates methods of using compounds for this purpose that have 100 to 1000 times greater activity than DEET in interfering with olfactory responses in insects. Additionally, some of the compounds described possess better selectivity than DEET.

In one aspect, the invention relates to method for interfering with the ability of an insect to detect odors, the method comprising exposing said insect to an olfactory-disrupting concentration of a compound of formula (a) below:

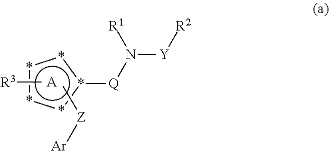

wherein
A is a five-membered, aromatic heterocycle in which one, two or three of the vertices marked by asterisks are heteroatoms chosen from nitrogen, oxygen and sulfur, and the remaining vertices are carbon;
Ar is optionally substituted aryl or heteroaryl;
Z is chosen from a direct bond, —CH₂—, —O—, —CH₂CH₂—, —CH₂O— and —OCH₂—;
Q is chosen from —(CH₂)ₘ—, wherein one or more —CH₂— may be replaced by —C═O—, —SO₂—, —NH—, —S—, —Ar^{a}—, —CHOH— or —O—;
Ar^{a} is optionally substituted aryl or heteroaryl;
Y is chosen from —(CH₂)ₘ—, wherein one or more —CH₂— may be replaced by —C═O—, —SO₂—, —NH—, —S—, —CHOH— or —O—;
R¹ and R² are independently chosen from H, (C₁-C₁₀)hydrocarbon, (C₁-C₁₀)oxaalkyl, aryl, substituted aryl, heterocyclyl, substituted heterocyclyl, —(CH₂)ₘR¹⁰ and —(CH₂)—CHR¹¹—CH₂—O—CH₂R¹⁰;
or taken together R¹ and R² form a 4-7 membered saturated monocycle or a 9-10 membered bicycle in which the ring formed by R¹—N—Y—R² is saturated, said monocycle or bicycle optionally substituted with one or two substituents chosen independently from halogen, (C₁-C₁₀)hydrocarbon, —C═O—, (C₁-C₁₀)oxaalkyl and —C(═O)R¹⁴;
R³ represents one or two residues chosen from hydrogen, (C₁-C₆ hydrocarbon, —NR¹²R¹³ , phenyl, and phenyl substituted with one or two substituents chosen from halogen, (C₁-C₄)alkyl, halo(C₁-C₄)alkyl, (C₁-C₄)alkoxy, and halo(C₁-C₄)alkoxy;
R¹⁰ is chosen from —COO(C₁-C₄)alkyl and monocyclic heterocycle;
R¹¹ is chosen from H, (C₁-C₁₀)hydrocarbon and —OH;
R¹² is chosen from H and (C₁-C₁₀)hydrocarbon;
R¹³ is chosen from H, (C₁-C₁₀)hydrocarbon, or R¹² and R¹³, taken together with the nitrogen to which they are attached, form a five- or six-membered ring optionally substituted with one or two substituents chosen from halogen, (C₁-C₈)hydrocarbon, and halo(C₁-C₄)alkyl;
R¹⁴ is chosen from H, (C₁-C₆)alkyl, (C₁-C₆)alkoxy and phenyl optionally substituted with one or two substituents chosen from halogen, (C ₁-C₄) alkyl, halo(C₁-C₄)alkyl, (C₁-C₄)alkoxy, and halo(C₁-C₄)alkoxy; and
m is 1, 2, 3, 4 or 5.

In one aspect, the invention relates to method for interfering with the ability of an insect to detect odors, the method comprising exposing said insect to an olfactory-disrupting concentration of a compound of formula (b) below:

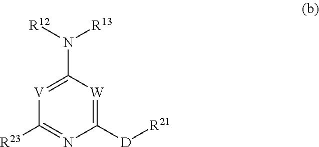

wherein V and W are independently chosen from N and CR¹²;
D is chosen from S, O and NR²⁴;
R²¹ is chosen from hydrogen, (C₁-C₁₀)hydrocarbon, —(CH₂)ₘCN, —(CH₂)ₘ—Ar^{a}, and —N═CH—Ar^{a};
R²³ is chosen from hydrogen, halogen, (C₁-C₁₀)hydrocarbon, halo(C₁-C₆)alkyl, (C₁-C₁₀)hydrocarbon-O— and —NR¹²R¹³; and
R²⁴ is chosen from H, (C₁-C₆)hydrocarbon and —CN.

In one aspect, the invention relates to method for interfering with the ability of an insect to detect odors, the method comprising exposing said insect to an olfactory-disrupting concentration of a compound of formula (c) below:

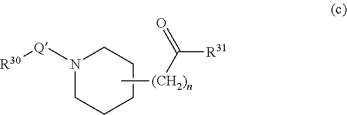

wherein
Q′ is chosen from —(CH₂)ₙC═O— and —(CH₂)ₙSO₂—, wherein the carbonyl or sulfonyl is the point of attachment to nitrogen;
n is chosen from 0, 1, 2, 3, 4 or 5;
R³⁰ is chosen from —Ar^{a}, —O—Ar^{a}, —S—Ar^{a}, and (C₁-C₁₀)hydrocarbon; and

R³¹ is chosen from —Ar, —NR¹²Ar, —NR¹²CH₂Ar, and

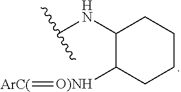

In one aspect, the invention relates to method for interfering with the ability of an insect to detect odors, the method comprising exposing said insect to an olfactory-disrupting concentration of a compound of formula (d) below:

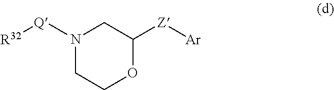

wherein
Z′ is chosen from, —CH₂—, —CH₂CH₂—, —CH₂O—, —CH₂OCH₂—, —CH₂CH₂CH₂— and —CH₂CH₂O—; and
R³² is chosen from —Ar^{a}, —OAr^{a}, —SAr^{a}, and —NHCOOCH₂Ar^{a}.

In one aspect, the invention relates to method for interfering with the ability of an insect to detect odors, the method comprising exposing said insect to an olfactory-disrupting concentration of a compound of formula (e) below:

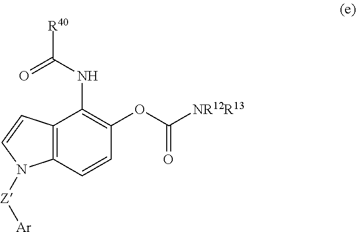

wherein R⁴⁰ is chosen from (C₁-C₁₀)hydrocarbon and an oxygenated (C₁-C₁₀)hydrocarbon.

In one aspect, the invention relates to a method for interfering with the ability of an insect to detect odors, the method comprising exposing said insect to an olfactory-disrupting concentration of a compound chosen from the compounds shown in the compound tables of the application.

In one aspect, the invention relates to an insect repellent composition suitable for topical application comprising a topically acceptable carrier and a compound described herein.

In one aspect, the invention relates to an insect repellent composition suitable for topical application comprising a topically acceptable carrier and a compound chosen from the compounds shown in the compound tables described herein.

### DETAILED DESCRIPTION OF THE INVENTION

Generally, one skilled in the art will recognize that the terms “Or83b” and “OR7”, as used herein, also can be referred to as “Orco,” See, for example, Vosshall, L. B., and Hansson, B. S., Chem. Senses, 2011 July; 36(6):497-8.

High-throughput screening experiments were performed to identify antagonists of the insect ORs, with the goal of using such antagonists to disrupt insect behavior, including host-seeking behavior of mosquitoes that spread infectious diseases to humans and attraction of insect pests to agricultural crops. A stable cell line was produced that expressed one of the ORs from the malaria mosquito (Anopheles gambiae) that is tuned to human odors. Using this cell line, a compound screen was completed that identified compounds whose action was tested on a number of diverse ORs from different insects and control receptors unrelated to the insect ORs. Compounds were identified with broad efficacy against numerous insect ORs but with some measure of selectivity for the insect ORs; thus, this suggests that these compounds may have efficacy as olfactory antagonists.

In one aspect, the invention relates to method for interfering with the ability of an insect to detect odors, the method comprising exposing said insect to an olfactory-disrupting concentration of a compound of formula (a):

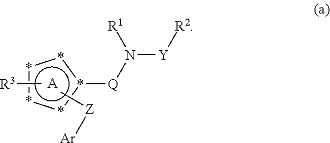

In some embodiments, A is a five-membered, aromatic heterocycle in which one, two or three of the vertices marked by asterisks are heteroatoms chosen from nitrogen, oxygen and sulfur, and the remaining vertices are carbon. Examples of A include, but are not necessarily limited to, oxazole, thiazole, isothiazole, oxadiazole, thiadiazole, isoxazole, imidazole, pyrazole or triazole.

In some embodiments, Ar is optionally substituted aryl or heteroaryl. In other embodiments, Ar is optionally substituted phenyl. In still other embodiments, Ar is optionally substituted 5- or 6-membered heteroaryl. In yet other embodiments, Ar is optionally substituted 8- to 10-membered bicyclic aryl or heteroaryl. In some embodiments, Ar is phenyl optionally substituted with 1, 2 or 3 substituents chosen in each instance from fluorine, chlorine and methyl.

In some embodiments, Ar^{a} is optionally substituted aryl or heteroaryl. In other embodiments, Ar^{a} is optionally substituted phenyl. In still other embodiments, Ar^{a} is optionally substituted 5- or 6-membered heteroaryl. For instance, Ar^{a} may be optionally substituted pyridine. In yet other embodiments, Ar^{a} is optionally substituted 8- to 10-membered bicyclic aryl or heteroaryl. In some embodiments, Ar^{a} is phenyl optionally substituted with 1, 2 or 3 substituents chosen in each instance from fluorine, chlorine and methyl.

In some embodiments, Z is chosen from a direct bond, —CH₂—, —O—, —CH₂CH₂—, —CH₂O— and —OCH₂—. In certain embodiments, Z is either a direct bond or —CH₂—.

In some embodiments, m may be 1, 2 3, 4 or 5.

In some embodiments, Q is chosen from —(CH₂)ₘ—, wherein one or more —CH₂— may be replaced by —C═O—, —SO₂—, —NH—, —S—, —Ar^{a}—, —CHOH— or —O—. Non-limiting examples of Q in the embodiments of the invention include —C(═O)—, —S(CH₂)Ar^{a}C(═O)—, —CH₂—, —(CH₂)₂C═O—, —(CH₂)C═O—, —(CH₂)S—, —S(CH₂)— and —S(CH₂)₁₋₃C(═O)—. In certain embodiments of the invention, Q is —CH₂—.

In some embodiments, Y is chosen from —(CH₂)ₘ—, wherein one or more —CH₂— may be replaced by —C═O—, —SO₂—, —NH—, —S—, —CHOH— or —O—. Non-limiting examples of Y in the embodiments of the invention include —C(═O)—, —CH₂CHOH(CH₂)O(CH₂)— and —CH₂—.

In some embodiments of the invention, at least one of -Q- and —YR² contains oxygen.

In some embodiments, R¹ may be chosen from H, (C₁-C₁₀)hydrocarbon, (C₁-C₁₀)oxaalkyl, aryl, substituted aryl, heterocyclyl, substituted heterocyclyl, —(CH₂)ₘR¹⁰ and —(CH₂)—CHR¹¹—CH₂—O—CH₂R¹⁰. In some embodiments, R¹ may be chosen from H, (C₁-C₈)hydrocarbon, (C₁-C₆)oxaalkyl, —(CH₂)furanyl and —(CH₂)tetrahydrofuranyl.

In some embodiments, R² may be chosen from H, (C₁-C₁₀)hydrocarbon, (C₁-C₁₀)oxaalkyl, aryl, substituted aryl, heterocyclyl, substituted heterocyclyl, —(CH₂)ₘR¹° and —(CH₂)—CHR¹¹—CH₂—O—CH₂R¹⁰.

In some embodiments, R¹⁰ is chosen from —COO(C₁-C₄)alkyl and monocyclic heterocycle. In some embodiments, R¹¹ is chosen from H, (C₁-C₁₀)hydrocarbon and —OH.

Non-limiting examples of R² in the embodiments of the invention include tetrahydroquinoline, furanyl, methylenedioxyphenyl, optionally substituted phenyl, optionally substituted pyridine, (C₁-C₈)hydrocarbon and (C₁-C₆)oxaalkyl. Non-limiting examples of optional substituents on a phenyl or pyridine include halogen and (C₁-C₆)oxaalkyl.

In some embodiments of the invention, R¹ and R² form a 4-7 membered saturated monocycle or a 9-10 membered bicycle in which the ring formed by R¹—N—Y—R² is saturated. In some embodiments, R¹NYR² forms an optionally substituted monocycle or bicycle chosen from pyrrolidine, piperidine, piperazine, morpholine, tetrahydroquinoline, tetrahydroisoquinoline, indoline and isoindoline. In some embodiments, the monocycle or bicycle is optionally substituted with one or two substituents chosen independently from halogen, (C₁-C₁₀)hydrocarbon, —C═O—, (C₁-C₁₀)oxaalkyl and —C(═O)R¹⁴.

In some embodiments, R¹⁴ is chosen from H, (C₁-C₆) alkyl, (C₁-C₆)alkoxy and phenyl optionally substituted with one or two substituents chosen from halogen, (C₁-C₄)alkyl, halo(C₁-C₄)alkyl, (C₁-C₄)alkoxy, and halo(C₁-C₄)alkoxy.

In some embodiments of the invention, Y is —C(═O)— and R² is chosen from (C₁-C₆) alkyl, optionally substituted phenyl and furanyl.

In some embodiments, R³ represents one or two residues chosen from hydrogen, (C₁-C₈) hydrocarbon, —NR¹²R¹³ , phenyl, and phenyl substituted with one or two substituents chosen from halogen, (C₁-C₄)alkyl, halo(C₁-C₄)alkyl, (C₁-C₄)alkoxy, and halo(C₁-C₄)alkoxy. In some embodiments, R³ is chosen from hydrogen, methyl, ethyl, —NR¹²R¹³, and phenyl optionally substituted with halogen, methyl and/or methoxy.

In some embodiments, R¹² and R¹³ are each independently chosen from H and (C₁-C₁₀)hydrocarbon. In other embodiments, R¹² and R¹³, taken together with the nitrogen to which they are attached, form a five- or six-membered ring optionally substituted with one or two substituents chosen from halogen, (C₁-C₈)hydrocarbon, (C₁-C₄)alkyl, (C₁-C₄)alkoxy, halo(C₁-C₄)alkoxy and halo(C₁-C₄)alkyl. This ring may additionally include other heteroatoms, such as would be found in morpholine or thiomorpholine.

In some embodiments, the compound is of formula:

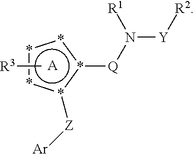

In some of these embodiments, A may be oxazole, thiazole, isothiazole, oxadiazole, thiadiazole, isoxazole, imidazole, pyrazole or triazole. In certain embodiments, A is selected from isoxazole, imidazole, pyrazole and triazole.

In some embodiments, the compound is of formula:

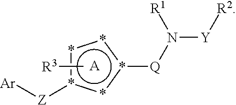

In some of these embodiments, A is pyrazole. In some embodiments, Q is —C(═O)— and Z is a direct bond.

In some embodiments, the compound is of formula

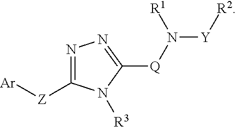

In some of these embodiments, Q is —S(CH₂)ₘC(═O)— or —S(CH₂)Ar^{a}C(═O)—, and R³ and Ar are both optionally substituted phenyl.

In some embodiments, the compound is of formula

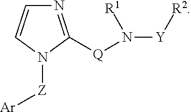

In some of these embodiments, Z is CH₂; Ar is phenyl optionally substituted with halogen; Q is CH₂ or C═O; Y is CH₂ or C═O; R¹ is (C₁-C₁₀)hydrocarbon; and R² is (C₁-C₁₀)hydrocarbon or phenyl optionally substituted with halogen.

In one aspect, the invention relates to method for interfering with the ability of an insect to detect odors, the method comprising exposing said insect to an olfactory-disrupting concentration of a compound of formula (b):

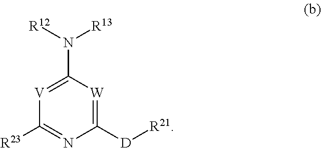

In one embodiment, V and W are both N.

In some of these embodiments, D is O. In other embodiments, D is NR²⁴, and R²⁴ is H. In still other embodiments, D is NR²⁴, and R²⁴ is (C₁-C₆)hydrocarbon. In yet other embodiments, D is NR²⁴, and R²⁴ is —CN.

In some embodiments, V is CR¹² and W is N. In some of these embodiments, D is S. In some embodiments, R¹² is H. In other embodiments, R¹² is (C₁-C₆)hydrocarbon. For instance, in some of these embodiments, R¹² is methyl or ethyl.

In some embodiments, the R¹² and R¹³ on N are each selected from H and (C₁-C₆)alkyl. For instance, in some embodiments, R¹² and R¹³ are each methyl. In other embodiments, R¹² is hydrogen and R¹³ is methyl, ethyl or isopropyl. In still other embodiments, R¹² is methyl and R¹³ is cyclohexyl. In some embodiments, NR¹²R¹³ is an unsaturated heterocycle. For instance, NR¹²R¹³ may be piperidine, pyrrolidine or morpholino.

In some embodiments, R²³ is chosen from hydrogen, halogen, (C₁-C₁₀)hydrocarbon, halo(C₁-C₆)alkyl, and (C₁-C₁₀)hydrocarbon-O—. For instance, R²³ may be hydrogen, methyl, ethyl or methoxy. In other embodiments, R²³ is —NR¹²R¹³. For instance, R²³ may be morpholino.

In some embodiments, D is NR²⁴ or S; R²⁴ is H or CH³ ; R²¹ is H or (C₁-C₁₀)hydrocarbon; and R¹² and R¹³ are each independently selected from H and (C₁-C₁₀)hydrocarbon.

In one aspect, the invention relates to method for interfering with the ability of an insect to detect odors, the method comprising exposing said insect to an olfactory-disrupting concentration of a compound of formula (c):

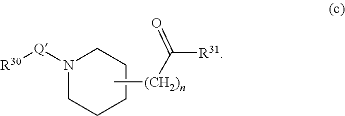

In some embodiments, Q′ is chosen from —(CH₂)ₙC═O— and —(CH₂)ₙSO₂—, wherein the carbonyl or sulfonyl is the point of attachment to nitrogen, and n is 0, 1, 2, 3, 4 or 5. In some embodiments, Q′ is —(CH₂)ₙC═O— and n is 0, 1 or 2.

In some embodiments, R³° is chosen from —Ar^{a}, —O—Ar^{a}, —S—Ar^{a}, and (C₁-C₁₀)hydrocarbon. In some embodiments, R³⁰ is chosen from —Ar^{a}, —OAr^{a} and —SAr^{a}, and Ar^{a} is optionally substituted phenyl. These substituents may include halogen, (C₁-C₁₀)hydrocarbon, (C₁-C₁₀)hydrocarbon wherein one carbon and its hydrogens may be substituted by oxygen, halo(C₁-C₁₀)alkyl, and monocyclic N-linked heterocycle.

In some embodiments, R³¹ is chosen from —Ar, —NR¹²Ar, —NR¹²CH₂Ar, and

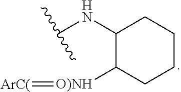

In some of these embodiments, Ar is optionally substituted phenyl.

In some embodiments, formula (c) is of formula

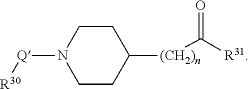

In other embodiments, formula (c) is of formula

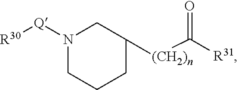

and n is zero for (CH₂)ₙ.

In one aspect, the invention relates to method for interfering with the ability of an insect to detect odors, the method comprising exposing said insect to an olfactory-disrupting concentration of a compound of formula (d):

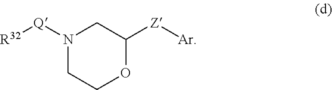

In some embodiments, Z′ is chosen from , —CH₂—, —CH₂CH₂—, —CH₂O—, —CH₂OCH₂—, —CH₂CH₂CH₂— and —CH₂CH₂O—. In other embodiments, Z′ is chosen from —CH₂—, —CH₂O—, and —CH₂OCH₂—.

In some embodiments, R³² is chosen from —Ar^{a}, —OAr^{a}, —SAr^{a}, and —NHCOOCH₂Ar^{a}. In other embodiments, R³² is —Ar^{a}. In still other embodiments, —Ar^{a} is chosen from optionally substituted phenyl and optionally substituted pyridine. In some cases, the substituents are selected from —N[(C₁-C₆)hydrocarbon]₂, halogen, cyano and methoxy.

In some embodiments, Q′ is chosen from —(CH₂)ₙC═O— and —(CH₂)ₙSO₂—, wherein the carbonyl or sulfonyl is the point of attachment to nitrogen, and n is 0, 1, 2, 3, 4 or 5. In some embodiments, Q′ is —(CH₂)ₙC═O— and n is 0, 1 or 2.

In some embodiments, Ar is optionally substituted aryl or heteroaryl. In other embodiments, Ar is optionally substituted phenyl. In other embodiments, the optional substituents are selected from halogen and methyl.

In one aspect, the invention relates to method for interfering with the ability of an insect to detect odors, the method comprising exposing said insect to an olfactory-disrupting concentration of a compound of formula (e):

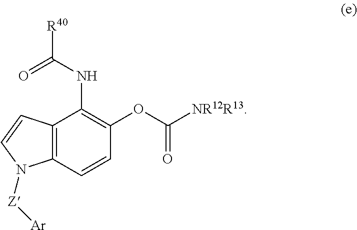

In some embodiments, Z′ is chosen from —CH₂—, —CH₂CH₂—, —CH₂O—, —CH₂OCH₂—, —CH₂CH₂CH₂— and —CH₂CH₂O—. In some embodiments, Z′ is —CH₂—.

In some embodiments, R¹² and R¹³ are chosen independently from H and (C₁-C₁₀)hydrocarbon. In some embodiments, R¹² and R¹³, taken together with the nitrogen to which they are attached, form a five- or six-membered ring optionally substituted with one or two substituents chosen from halogen, (C₁-C₈)hydrocarbon, and halo(C₁-C₄)alkyl. In some embodiments, R¹² and R¹³ are each independently (C₁-C₄) alkyl.

In some embodiments, R⁴⁰ is selected from (C₁-C₁₀)hydrocarbon and an oxygenated (C₁-C₁₀) hydrocarbon. In other embodiments, R⁴⁰ is selected from —(C₁-C₄)hydrocarbon-C(═O)—O—(C₁-C₄)hydrocarbon, —(C₁-C₄)hydrocarbon-O—C(═O)—(C₁-C₄)hydrocarbon, and (C₁-C₆)hydrocarbon. In still other embodiments, R⁴⁰ is selected from —(C₁-C₄)alkyl-C(═O)—O—CH₃, —(C₁-C₄)alkyl-O—C(═O)CH₃, and (C₁-C₆)alkyl.

In one aspect, the invention relates to a method for interfering with the ability of an insect to detect odors, the method comprising exposing said insect to an olfactory-disrupting concentration of a compound chosen from the compounds shown in the compound tables of the application.

In some embodiments, a compound described herein is included in an insect repellent composition suitable for topical application in combination with a topically acceptable carrier.

In some embodiments, a compound chosen from the compounds shown in the compound tables of the application is included in an insect repellent composition suitable for topical application in combination with a topically acceptable carrier.

Definitions

Throughout this specification the terms and substituents retain their definitions.

Alkyl is intended to include linear, branched, or cyclic hydrocarbon structures and combinations thereof. A combination would be, for example, cyclopropylmethyl. Lower alkyl refers to alkyl groups of from 1 to 6 carbon atoms. Examples of lower alkyl groups include methyl, ethyl, propyl, isopropyl, cyclopropyl, butyl, s-and t-butyl, cyclobutyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

Alkoxy or alkoxyl refers to groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Oxaalkyl refers to alkyl residues (including cycloalkyls) in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples include ethylaminoethyl and methylthiopropyl.

Hydrocarbon means a linear, branched, or cyclic residue comprised of hydrogen and carbon as the only elemental constituents and includes alkyl, cycloalkyl, polycycloalkyl, alkenyl, alkynyl, aryl and combinations thereof. Examples include, but are not limited to, e.g., benzyl, phenyl, phenethyl, cyclohexylmethyl and naphthylethyl.

Heterocycle means a cycloalkyl or aryl residue in which one to four of the carbons is replaced by a heteroatom such as oxygen, nitrogen or sulfur. Heteroaryls form a subset of heterocycles. Examples of heterocycles that fall within the scope of the invention include, but are not limited to, e.g., pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), methylenedioxyphenyl, ethylenedioxyphenyl, tetrazole, morpholine, thiazole, pyridine, pyridazine, pyrimidine, thiophene, furan, oxazole, oxazoline, isoxazole, dioxane, tetrahydrofuran and the like.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, but are not limited to, e.g., benzene, naphthalene, indane, tetralin, and fluorene and the 5- to 10-membered aromatic heterocyclic rings include, but are not limited to, e.g., imidazole, pyridine, indole, thiophene, benzopyranone, thiazole, furan, benzimidazole, quinoline, isoquinoline, quinoxaline, pyrazolone, pyrrolimidazole, oxadiazole, thiadiazole, pyrimidine, pyrazine, isoindole, benzothiophene, indazole, benzthiazole, cinnoline, phthalazine, quinazoline, pteridine, tetrazole and pyrazole. As used herein aryl and heteroaryl refer to residues in which one or more rings are aromatic, but not all need be.

Arylalkyl means an alkyl residue attached to an aryl ring. Examples are benzyl, phenethyl and the like. Heteroarylalkyl means an alkyl residue attached to a heteroaryl ring. Examples include, but are not limited to, e.g., pyridinylmethyl, pyrimidinylethyl and the like.

The term “halogen” means fluorine, chlorine, bromine or iodine. In one embodiment, halogen may be fluorine or chlorine.

The terms “haloalkyl” and “haloalkoxy” mean alkyl or alkoxy, respectively, substituted with one or more halogen atoms.

Substituted alkyl, aryl, cycloalkyl, heterocyclyl etc. refer to alkyl, aryl, cycloalkyl, or heterocyclyl wherein up to four H atoms in each residue are replaced with halogen, haloalkyl, alkyl, acyl, alkoxyalkyl, allyloxy, hydroxyloweralkyl, phenyl, heteroaryl, benzenesulfonyl, hydroxy, loweralkoxy, haloalkoxy, carboxy, carboalkoxy (also referred to as alkoxycarbonyl), alkoxycarbonylamino, alkoxyaminocarbonyl, carboxamido (also referred to as alkylaminocarbonyl), cyano, carbonyl, acetoxy, nitro, amino, alkylamino, dialkylamino, mercapto, alkylthio, sulfoxide, sulfone, sulfonylamino, acylamino, amidino, aryl, benzyl, methyl-substituted benzyl, halo-substituted benzyl, arylcarbonyl (wherein a phenyl group may be substituted with (C₁-C₆)alkyl, (C₁-C₆)haloalkyl, and/or halogen), heterocyclyl, heterocyclylcarbonyl, phenoxy, benzyloxy, heteroaryloxy, hydroxyimino, alkoxyimino, oxaalkyl, aminosulfonyl, trityl, amidino, guanidino, ureido, and benzyloxy.

Some of the compounds described herein contain one or more asymmetric centers and may thus give rise to enantiomers, diastereomers, and other stereoisomeric forms that may be defined, in terms of absolute stereochemistry, as (R)— or (S)—. The present invention is meant to include all such possible isomers, as well as their racemic and optically pure forms. In general it has been found that the levo isomer of morphinans and benzomorphans is the more potent antinociceptive agent, while the dextro isomer may be useful as an antitussive or antispasmodic agent. Optically active (R)— and (S)— isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The compounds of the invention may be used in topical compositions. The topical compositions comprise a topically acceptable carrier and a compound as described above. For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

The topical carrier may include any substance capable of dispersing and maintaining contact between the active ingredients and the skin. The vehicle may be glycerin, alcohol or water based. Non-limiting examples of such vehicles include aloe vera, which is a gel base, together with ethanol, isopropyl alcohol, water, propylene glycol and a non-ionic surfactant such as laureth-4. Other water-based alcohol/glycerin vehicles and carriers are within the scope of the present invention. A typical water-based lotion will contain from 45 to 50 parts of glycerin, one to three parts Tween 80™, from 45 to 50 parts of water and from 1 to 50 parts of the compound of the invention.

Also included in the scope of the invention are ointments, emulsions or dispersions in which water, if present, is a minor constituent. Typical ointment formulation comprises from 90 to 98 parts of a mixture of petrolatum, mineral oil, mineral wax and wool wax alcohol, from 0.5 to 3 parts of a mixture of polyoxyethylene and sorbitan monooleate (Tween 80™), from 1 to 5 parts of water, and from 1 to 50 parts of the compound of the invention. Another suitable non-aqueous ointment can be prepared from 95 parts of liquid petrolatum USP, 5 parts polyethylene and from 1 to 50 parts of the compound of the invention. The resulting ointment spreads easily and has an even consistency over wide temperature extremes. It is, in addition, non-irritating and non-sensitizing.

Water based compositions may also be employed, in which case the compound of the invention will commonly be in solution, and the aqueous solution may, if desired, be thickened with a suitable gel to provide a less mobile composition. Such compositions are well known in the art.

Compounds

The following tables include compounds used in the methods of the instant invention.

| Compound ID | Structure |
|---|---|
| RU-0000619 | 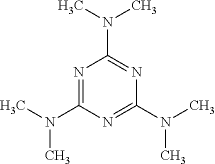 |
| RU-0001534 | 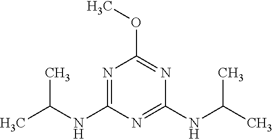 [SMILES: COc1nc(NC(C)C)nc(NC(C)C)n1] |
| RU-0006256 | 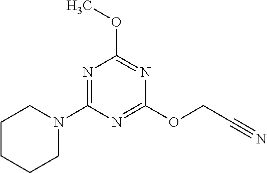 [SMILES: COc1nc(OCC#N)nc(N2CCCCC2)n1] |
| RU-0015806 | 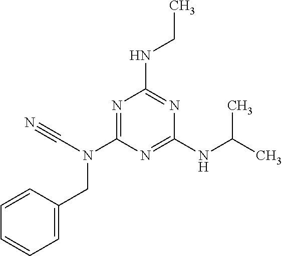 [SMILES: CCNc1nc(NC(C)C)nc(N(C#N)Cc2ccccc2)n1] |
| RU-0018338 | 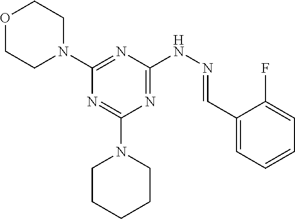 [SMILES: Fc1ccccc1/C=N/Nc1nc(N2CCCCC2)nc(N2CCOCC2)n1] |
| RU-0020050 | 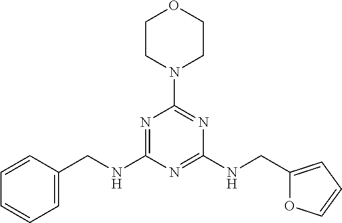 [SMILES: c1ccc(CNc2nc(NCc3ccco3)nc(N3CCOCC3)n2)cc1] |
| RU-0026074 | 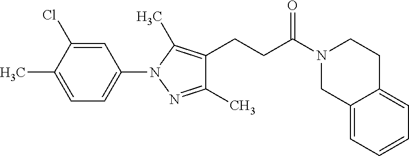 [SMILES: Cc1ccc(-n2nc(C)c(CCC(=O)N3CCc4ccccc4C3)c2C)cc1Cl] |
| RU-0028727 | 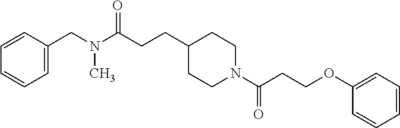 [SMILES: CN(Cc1ccccc1)C(=O)CCC1CCN(C(=O)CCOc2ccccc2)CC1] |
| RU-0029350 | 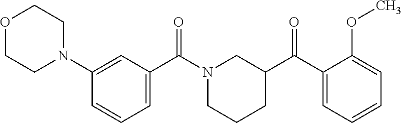 [SMILES: COc1ccccc1C(=O)C1CCCN(C(=O)c2cccc(N3CCOCC3)c2)C1] |
| RU-0029409 | 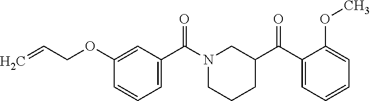 [SMILES: C=CCOc1cccc(C(=O)N2CCCC(C(=O)c3ccccc3OC)C2)c1] |
| RU-0030324 | 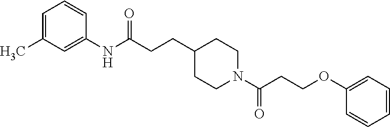 [SMILES: Cc1cccc(NC(=O)CCC2CCN(C(=O)CCOc3ccccc3)CC2)c1] |
| RU-0032455 | 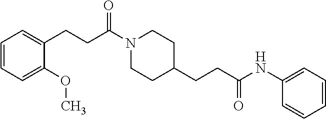 [SMILES: COc1ccccc1CCC(=O)N1CCC(CCC(=O)Nc2ccccc2)CC1] |
| RU-0032465 | 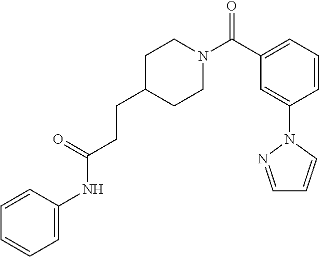 [SMILES: O=C(CCC1CCN(C(=O)c2cccc(-n3cccn3)c2)CC1)Nc1ccccc1] |
| RU-0032474 | 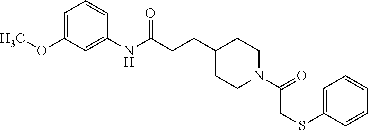 [SMILES: COc1cccc(NC(=O)CCC2CCN(C(=O)CSc3ccccc3)CC2)c1] |
| RU-0034108 | 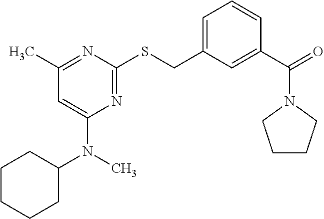 [SMILES: Cc1cc(N(C)C2CCCCC2)nc(SCc2cccc(C(=O)N3CCCC3)c2)n1] |
| RU-0036855 | 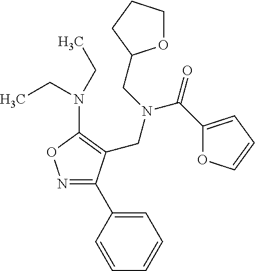 [SMILES: CCN(CC)c1onc(-c2ccccc2)c1CN(CC1CCCO1)C(=O)c1ccco1] |
| RU-0036875 | 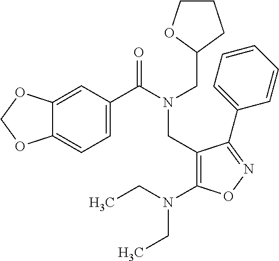 [SMILES: CCN(CC)c1onc(-c2ccccc2)c1CN(CC1CCCO1)C(=O)c1ccc2c(c1)OCO2] |
| RU-0036876 | 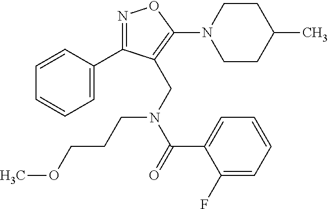 |
| RU-0036983 | 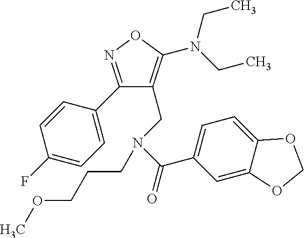 [SMILES: CCN(CC)c1onc(-c2ccc(F)cc2)c1CN(CCCOC)C(=O)c1ccc2c(c1)OCO2] |
| RU-0037003 | 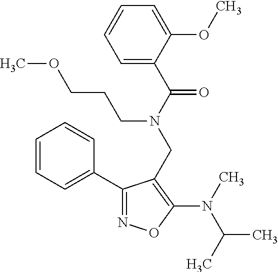 [SMILES: COCCCN(Cc1c(-c2ccccc2)noc1N(C)C(C)C)C(=O)c1ccccc1OC] |
| RU-0037275 | 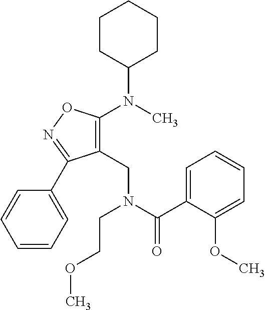 [SMILES: COCCN(Cc1c(-c2ccccc2)noc1N(C)C1CCCCC1)C(=O)c1ccccc1OC] |
| RU-0037278 | 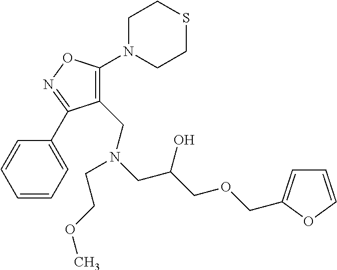 [SMILES: COCCN(Cc1c(-c2ccccc2)noc1N1CCSCC1)CC(O)COCc1ccco1] |
| RU-0037386 | 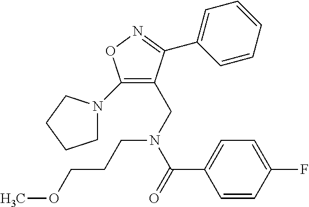 [SMILES: COCCCN(Cc1c(-c2ccccc2)noc1N1CCCC1)C(=O)c1ccc(F)cc1] |
| RU-0037434 | 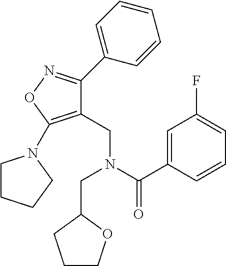 [SMILES: O=C(c1cccc(F)c1)N(Cc1c(-c2ccccc2)noc1N1CCCC1)CC1CCCO1] |
| RU-0038795 | 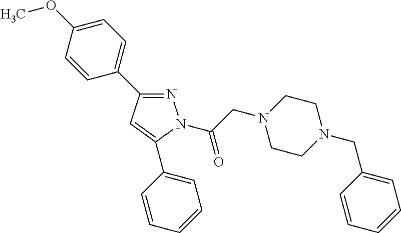 [SMILES: COc1ccc(-c2cc(-c3ccccc3)n(C(=O)CN3CCN(Cc4ccccc4)CC3)n2)cc1] |
| RU-0039123 | 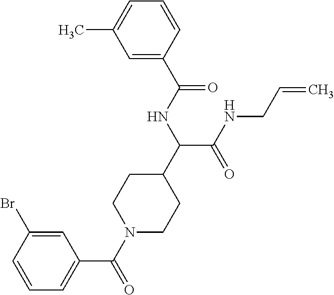 [SMILES: C=CCNC(=O)C(NC(=O)c1cccc(C)c1)C1CCN(C(=O)c2cccc(Br)c2)CC1] |
| RU-0045351 | 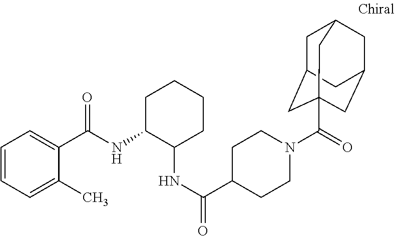 [SMILES: Cc1ccccc1C(=O)N[C@@H]1CCCCC1NC(=O)C1CCN(C(=O)C23CC4CC(CC(C4)C2)C3)CC1] |
| RU-0045678 | 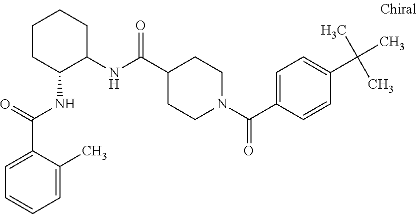 [SMILES: Cc1ccccc1C(=O)N[C@@H]1CCCCC1NC(=O)C1CCN(C(=O)c2ccc(C(C)(C)C)cc2)CC1] |
| RU-0045691 | 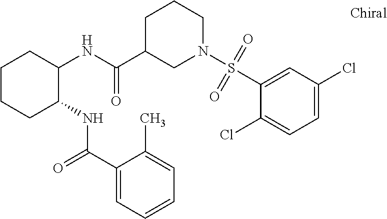 [SMILES: Cc1ccccc1C(=O)N[C@@H]1CCCCC1NC(=O)C1CCCN(S(=O)(=O)c2cc(Cl)ccc2Cl)C1] |
| RU-0045756 | 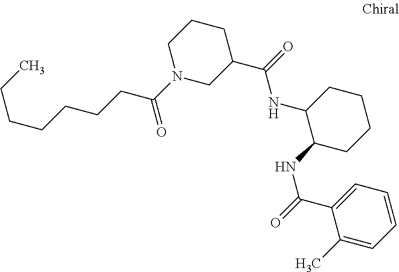 [SMILES: CCCCCCCC(=O)N1CCCC(C(=O)NC2CCCC[C@H]2NC(=O)c2ccccc2C)C1] |
| RU-0045778 | 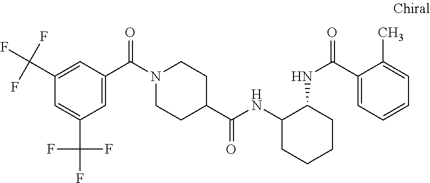 [SMILES: Cc1ccccc1C(=O)N[C@@H]1CCCCC1NC(=O)C1CCN(C(=O)c2cc(C(F)(F)F)cc(C(F)(F)F)c2)CC1] |
| RU-0047281 | 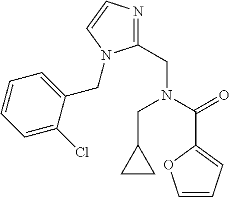 [SMILES: O=C(c1ccco1)N(Cc1nccn1Cc1ccccc1Cl)CC1CC1] |
| RU-0047568 | 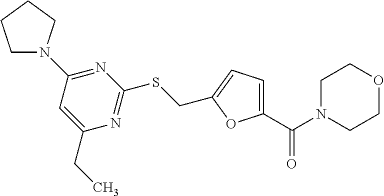 [SMILES: CCc1cc(N2CCCC2)nc(SCc2ccc(C(=O)N3CCOCC3)o2)n1] |
| RU-0047694 | 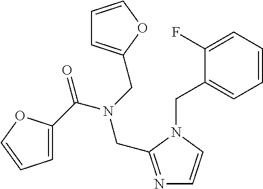 [SMILES: O=C(c1ccco1)N(Cc1ccco1)Cc1nccn1Cc1ccccc1F] |
| RU-0047765 | 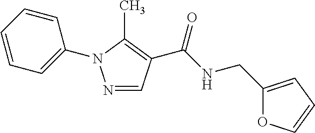 [SMILES: Cc1c(C(=O)NCc2ccco2)cnn1-c1ccccc1] |
| RU-0047851 | 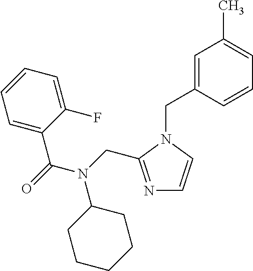 [SMILES: Cc1cccc(Cn2ccnc2CN(C(=O)c2ccccc2F)C2CCCCC2)c1] |
| RU-0047955 | 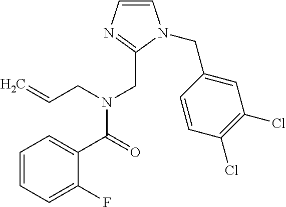 [SMILES: C=CCN(Cc1nccn1Cc1ccc(Cl)c(Cl)c1)C(=O)c1ccccc1F] |
| RU-0048001 | 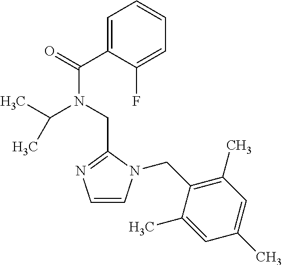 [SMILES: Cc1cc(C)c(Cn2ccnc2CN(C(=O)c2ccccc2F)C(C)C)c(C)c1] |
| RU-0048046 | 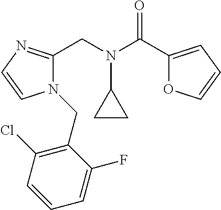 [SMILES: O=C(c1ccco1)N(Cc1nccn1Cc1c(F)cccc1Cl)C1CC1] |
| RU-0048054 | 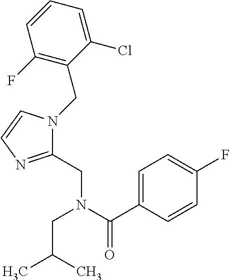 [SMILES: CC(C)CN(Cc1nccn1Cc1c(F)cccc1Cl)C(=O)c1ccc(F)cc1] |
| RU-0048066 | 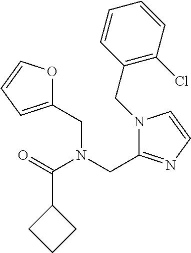 [SMILES: O=C(C1CCC1)N(Cc1ccco1)Cc1nccn1Cc1ccccc1Cl] |
| RU-0048504 | 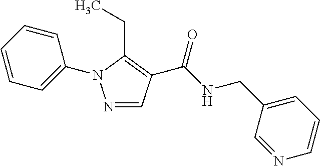 [SMILES: CCc1c(C(=O)NCc2cccnc2)cnn1-c1ccccc1] |
| RU-0048608 | 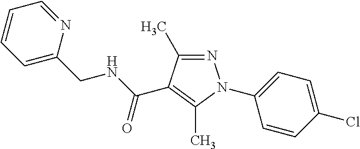 [SMILES: Cc1nn(-c2ccc(Cl)cc2)c(C)c1C(=O)NCc1ccccn1] |
| RU-0050325 | 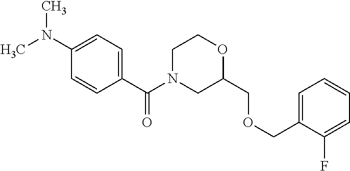 [SMILES: CN(C)c1ccc(C(=O)N2CCOC(COCc3ccccc3F)C2)cc1] |
| RU-0050344 | 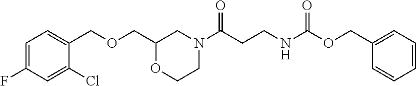 [SMILES: O=C(NCCC(=O)N1CCOC(COCc2ccc(F)cc2Cl)C1)OCc1ccccc1] |
| RU-0050372 | 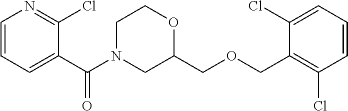 [SMILES: O=C(c1cccnc1Cl)N1CCOC(COCc2c(Cl)cccc2Cl)C1] |
| RU-0050384 | 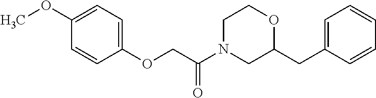 [SMILES: COc1ccc(OCC(=O)N2CCOC(Cc3ccccc3)C2)cc1] |
| RU-0050390 | 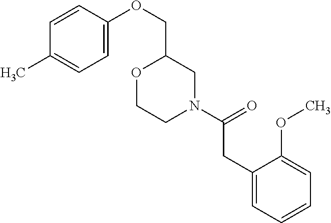 |
| RU-0050393 | 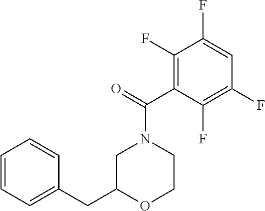 [SMILES: O=C(c1c(F)c(F)cc(F)c1F)N1CCOC(Cc2ccccc2)C1] |
| RU-0050398 | 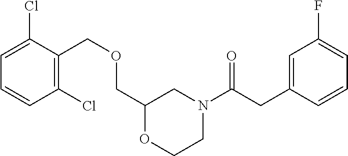 [SMILES: O=C(Cc1cccc(F)c1)N1CCOC(COCc2c(Cl)cccc2Cl)C1] |
| RU-0050405 | 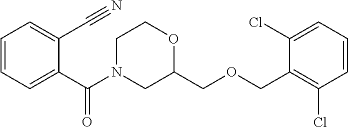 [SMILES: N#Cc1ccccc1C(=O)N1CCOC(COCc2c(Cl)cccc2Cl)C1] |
| RU-0051926 | 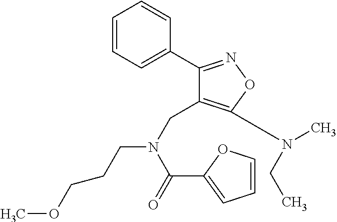 |
| RU-0053556 | 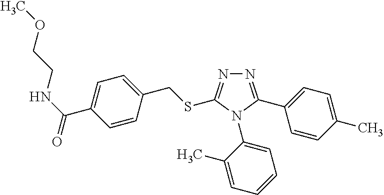 |
| RU-0055322 | 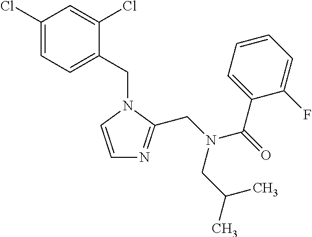 [SMILES: CC(C)CN(Cc1nccn1Cc1ccc(Cl)cc1Cl)C(=O)c1ccccc1F] |
| RU-0055364 | 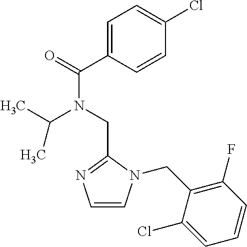 [SMILES: CC(C)N(Cc1nccn1Cc1c(F)cccc1Cl)C(=O)c1ccc(Cl)cc1] |
| RU-0058232 | 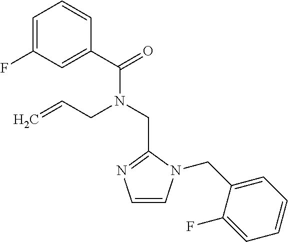 [SMILES: C=CCN(Cc1nccn1Cc1ccccc1F)C(=O)c1cccc(F)c1] |
| RU-0058298 | 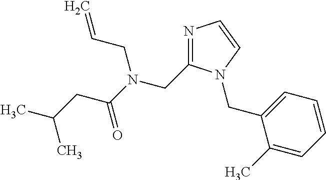 [SMILES: C=CCN(Cc1nccn1Cc1ccccc1C)C(=O)CC(C)C] |
| RU-0058550 | 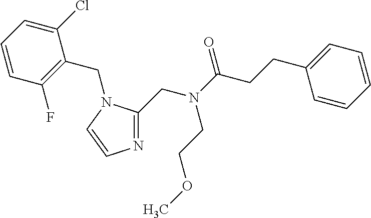 [SMILES: COCCN(Cc1nccn1Cc1c(F)cccc1Cl)C(=O)CCc1ccccc1] |
| RU-0058964 | 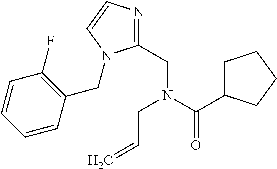 [SMILES: C=CCN(Cc1nccn1Cc1ccccc1F)C(=O)C1CCCC1] |
| RU-0071273 | 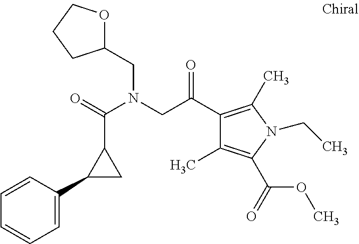 [SMILES: CCn1c(C)c(C(=O)CN(CC2CCCO2)C(=O)C2C[C@H]2c2ccccc2)c(C)c1C(=O)OC] |
| RU-0071628 | 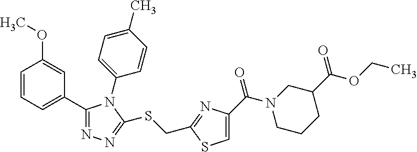 [SMILES: CCOC(=O)C1CCCN(C(=O)c2csc(CSc3nnc(-c4cccc(OC)c4)n3-c3ccc(C)cc3)n2)C1] |
| RU-0071789 | 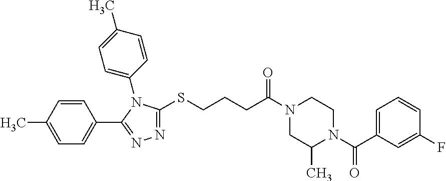 [SMILES: Cc1ccc(-c2nnc(SCCCC(=O)N3CCN(C(=O)c4cccc(F)c4)C(C)C3)n2-c2ccc(C)cc2)cc1] |
| RU-0071818 | 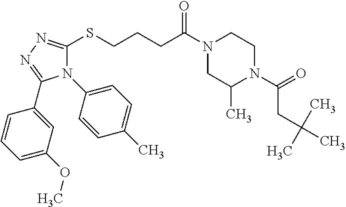 [SMILES: COc1cccc(-c2nnc(SCCCC(=O)N3CCN(C(=O)CC(C)(C)C)C(C)C3)n2-c2ccc(C)cc2)c1] |
| RU-0071828 | 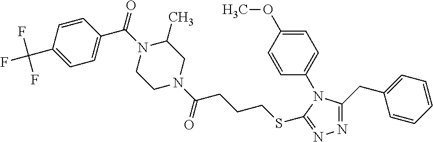 [SMILES: COc1ccc(-n2c(Cc3ccccc3)nnc2SCCCC(=O)N2CCN(C(=O)c3ccc(C(F)(F)F)cc3)C(C)C2)cc1] |
| RU-0072462 | 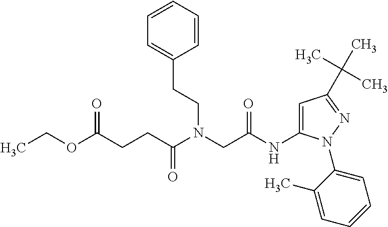 [SMILES: CCOC(=O)CCC(=O)N(CCc1ccccc1)CC(=O)Nc1cc(C(C)(C)C)nn1-c1ccccc1C] |
| RU-0075445 | 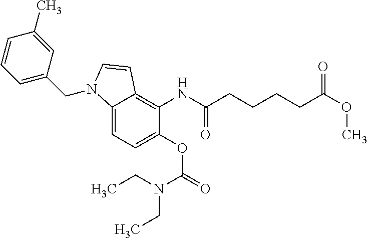 [SMILES: CCN(CC)C(=O)Oc1ccc2c(ccn2Cc2cccc(C)c2)c1NC(=O)CCCCC(=O)OC] |
| RU-0075446 | 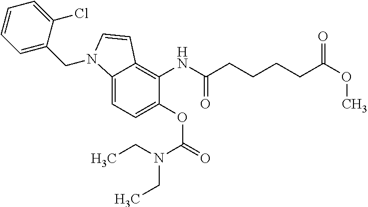 [SMILES: CCN(CC)C(=O)Oc1ccc2c(ccn2Cc2ccccc2Cl)c1NC(=O)CCCCC(=O)OC] |
| RU-0075468 | 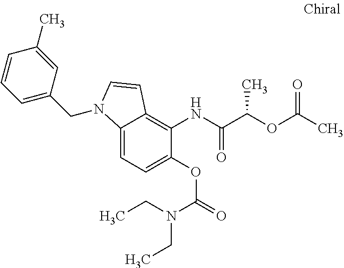 [SMILES: CCN(CC)C(=O)Oc1ccc2c(ccn2Cc2cccc(C)c2)c1NC(=O)[C@H](C)OC(C)=O] |
| RU-0075475 | 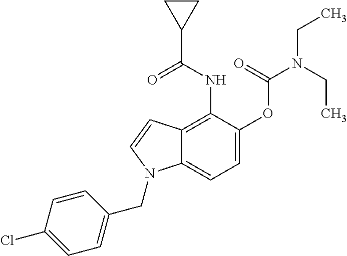 [SMILES: CCN(CC)C(=O)Oc1ccc2c(ccn2Cc2ccc(Cl)cc2)c1NC(=O)C1CC1] |
| RU-0001327 | 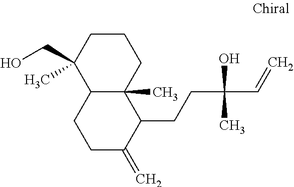 [SMILES: C=C[C@@](C)(O)CCC1C(=C)CCC2[C@@](C)(CO)CCC[C@]12C] |
| RU-0001617 | 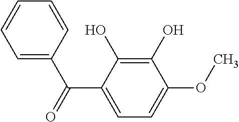 [SMILES: COc1ccc(C(=O)c2ccccc2)c(O)c1O] |
| RU-0023245 | 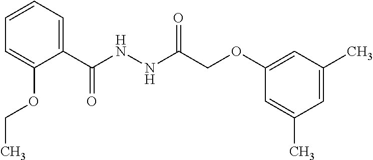 [SMILES: CCOc1ccccc1C(=O)NNC(=O)COc1cc(C)cc(C)c1] |
| RU-0025771 | 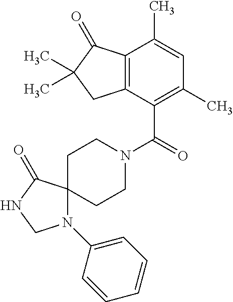 [SMILES: Cc1cc(C)c2c(c1C(=O)N1CCC3(CC1)C(=O)NCN3c1ccccc1)CC(C)(C)C2=O] |
| RU-0026167 | 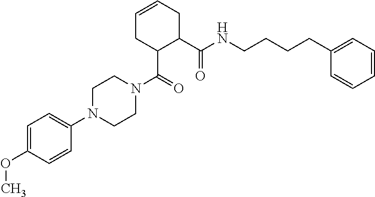 [SMILES: COc1ccc(N2CCN(C(=O)C3CC=CCC3C(=O)NCCCCc3ccccc3)CC2)cc1] |
| RU-0026507 | 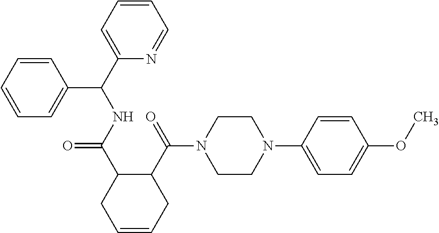 [SMILES: COc1ccc(N2CCN(C(=O)C3CC=CCC3C(=O)NC(c3ccccc3)c3ccccn3)CC2)cc1] |
| RU-0028029 | 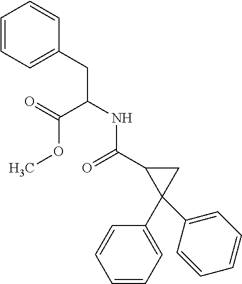 [SMILES: COC(=O)C(Cc1ccccc1)NC(=O)C1CC1(c1ccccc1)c1ccccc1] |
| RU-0031879 | 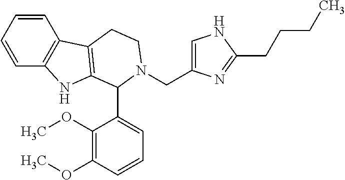 [SMILES: CCCCc1nc(CN2CCc3c([nH]c4ccccc34)C2c2cccc(OC)c2OC)c[nH]1] |
| RU-0035287 | 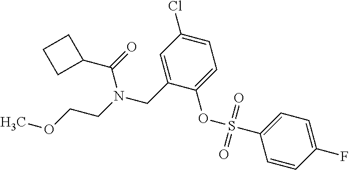 [SMILES: COCCN(Cc1cc(Cl)ccc1OS(=O)(=O)c1ccc(F)cc1)C(=O)C1CCC1] |
| RU-0036264 | 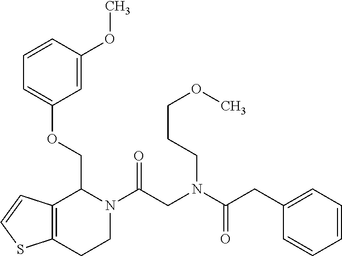 [SMILES: COCCCN(CC(=O)N1CCc2sccc2C1COc1cccc(OC)c1)C(=O)Cc1ccccc1] |
| RU-0036294 | 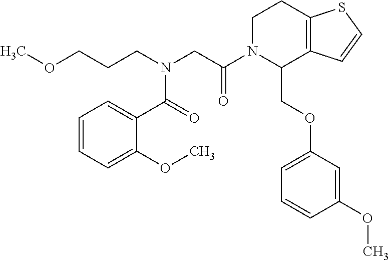 |
| RU-0043595 | 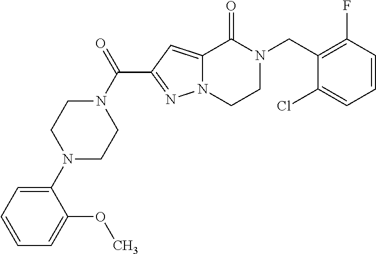 [SMILES: COc1ccccc1N1CCN(C(=O)c2cc3n(n2)CCN(Cc2c(F)cccc2Cl)C3=O)CC1] |
| RU-0045115 | 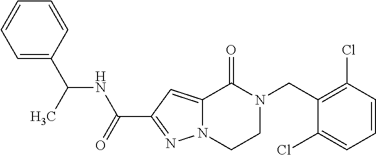 [SMILES: CC(NC(=O)c1cc2n(n1)CCN(Cc1c(Cl)cccc1Cl)C2=O)c1ccccc1] |
| RU-0046426 | 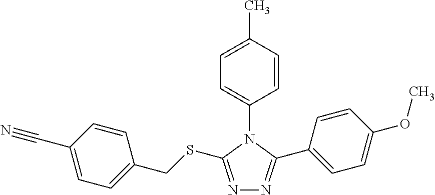 [SMILES: COc1ccc(-c2nnc(SCc3ccc(C#N)cc3)n2-c2ccc(C)cc2)cc1] |
| RU-0047750 | 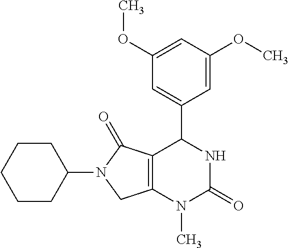 [SMILES: COc1cc(OC)cc(C2NC(=O)N(C)C3=C2C(=O)N(C2CCCCC2)C3)c1] |
| RU-0049030 | 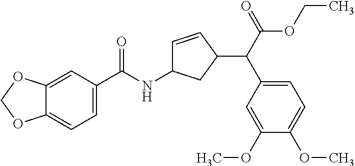 [SMILES: CCOC(=O)C(c1ccc(OC)c(OC)c1)C1C=CC(NC(=O)c2ccc3c(c2)OCO3)C1] |
| RU-0051652 | 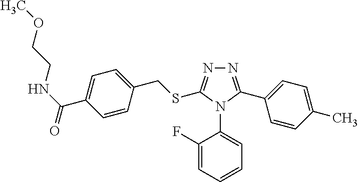 |
| RU-0052340 | 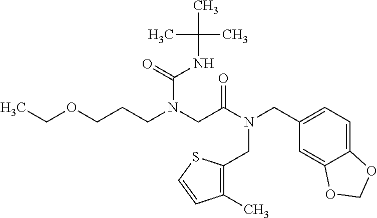 [SMILES: CCOCCCN(CC(=O)N(Cc1ccc2c(c1)OCO2)Cc1sccc1C)C(=O)NC(C)(C)C] |
| RU-0052360 | 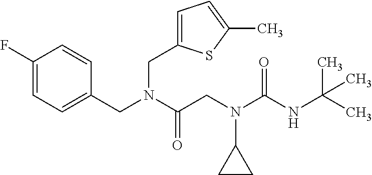 [SMILES: Cc1ccc(CN(Cc2ccc(F)cc2)C(=O)CN(C(=O)NC(C)(C)C)C2CC2)s1] |
| RU-0069950 | 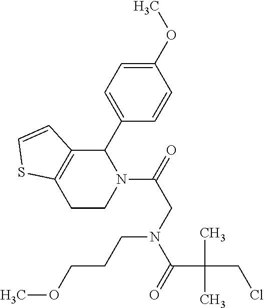 [SMILES: COCCCN(CC(=O)N1CCc2sccc2C1c1ccc(OC)cc1)C(=O)C(C)(C)CCl] |
| RU-0072008 | 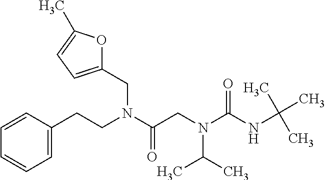 [SMILES: Cc1ccc(CN(CCc2ccccc2)C(=O)CN(C(=O)NC(C)(C)C)C(C)C)o1] |
| RU-0072535 | 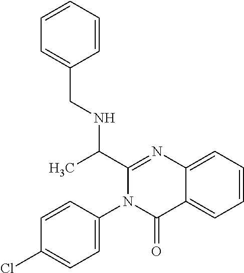 [SMILES: CC(NCc1ccccc1)c1nc2ccccc2c(=O)n1-c1ccc(Cl)cc1] |
| RU-0074560 | 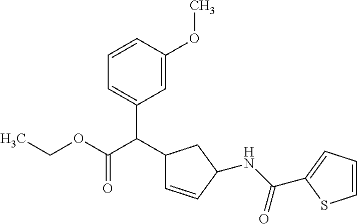 [SMILES: CCOC(=O)C(c1cccc(OC)c1)C1C=CC(NC(=O)c2cccs2)C1] |
| RU-0076832 | 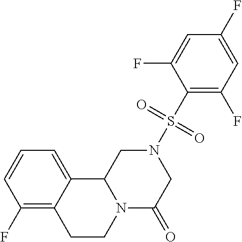 [SMILES: O=C1CN(S(=O)(=O)c2c(F)cc(F)cc2F)CC2c3cccc(F)c3CCN12] |
| RU-0077733 | 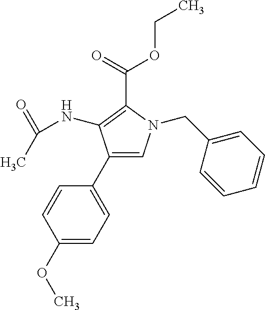 [SMILES: CCOC(=O)c1c(NC(C)=O)c(-c2ccc(OC)cc2)cn1Cc1ccccc1] |
| RU-0079120 | 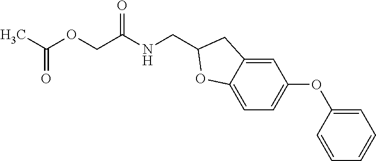 [SMILES: CC(=O)OCC(=O)NCC1Cc2cc(Oc3ccccc3)ccc2O1] |
| RU-I1 | 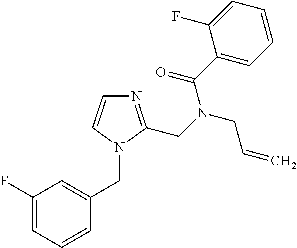 |
| RU-I2 | 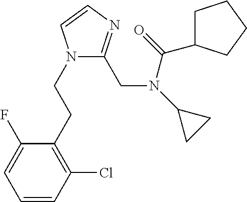 |
| RU-I3 | 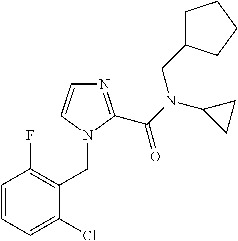 |
| RU-I4 | 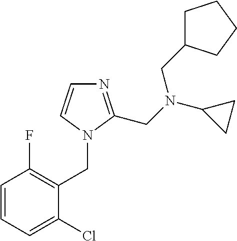 |
| RU-I5 | 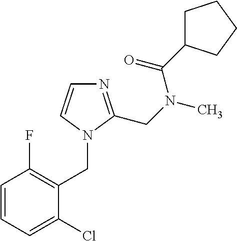 |
| RU-T1 | 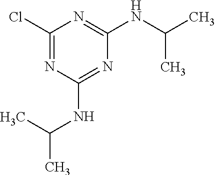 |
| RU-T2 | 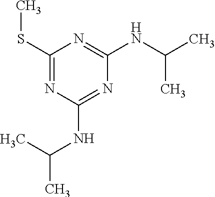 |
| RU-T3 | 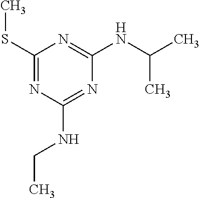 |
| RU-T4 | 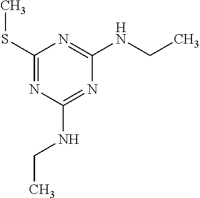 |
| RU-T5 | 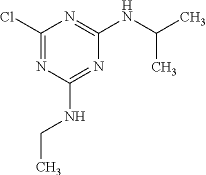 |
| RU-T6 | 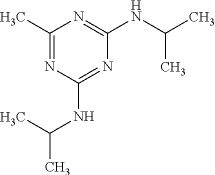 |
| RU-T7 | 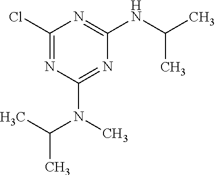 |
| RU-T8 | 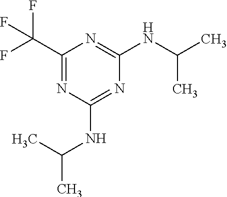 |
| RU-T9 | 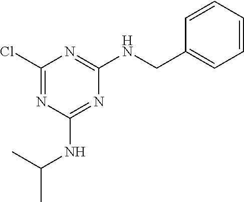 |

Assay Protocols

A. Molecular Biology

Full-length cDNAs for mosquito (Anopheles gambiae) odorant receptor (OR1, OR2, OR7, OR8, OR28), and fly (Drosophila melanogaster) odorant receptor (Or47a, Or83b), mouse TRP channel (TRPM8), and mouse serotonin receptor (5HT3) were obtained and subcloned into mammalian expression vectors. The following mammalian expression vectors were used:

pME18s-bla

This vector was constructed by inserting the blasticidin resistance gene derived from pcDNA6/His™ vector (Invitrogen) into the SspI site of pME18s with a blunt-ended ligation.

pME18s-puro

This vector was constructed by inserting the puromycin resistance gene from pPUR (BD Bioscience/Clontech) into the SspI site of pME18s with a blunt-ended ligation.

pcDNA5/FRT/TO

This vector was supplied by Invitrogen with a blasticidin resistance gene and was used without further modification.

pcDNA5/FRT/TO/IRES

This vector was constructed by adding an internal ribosome entry site (IRES) element from pIRES vector (Clontech) into the EcoRV-XhoI site of the pcDNA5/FRT/TO vector (Invitrogen).

The constructs were generated as follows:

| CONSTRUCT | VECTOR |
|---|---|
| OR1 | pME18s-bla |
| OR2 | pME18s-bla |
| OR7 | pME18s-puro |
| OR8 | pME18s-bla |
| OR28 | pME18s-bla |
| Or47/Or83b | pcDNA5/FRT/TO/IRES (5′ of IRES: Or47a; 3′ of IRES: |
| | Or83b) |
| TRPM8 | pME18s-puro |
| 5HT3 | pcDNA5/FRT/TO |

DNA was manipulated using standard molecular biology techniques. All plasmid junctions were sequenced to verify integrity prior to use in constructing cell lines.

B. Cell Culture

Human embryonic kidney 293T (HEK293T) or Flip-In T-Rex cells were grown at 37° C. in humidified air containing 5% carbon dioxide (CO₂) in Dulbecco's modified Eagle's medium (DMEM) supplemented with 10% fetal bovine serum (FBS) (heat-inactivated) and 100 units (U) of penicillin and 100 μg/mL of streptomycin.

Stable cell lines OR1/OR7, OR2/OR7, OR8/OR7, OR10/OR7, OR28/OR7 and TRPM8 were generated from HEK293T cells. HEK293T-based cell lines were cultured in DMEM supplemented with these additional antibiotics: 6 μg/mL blasticidin and 1 μg/mL puromycin, with the exception of the TRPM8 cell lines, which was cultured in DMEM supplemented with 1 μg/mL puromycin.

Stable cell lines Or47a/Or83b and 5HT3 were generated from Flip-In T-Rex cells. Flip-In T-Rex-based cell lines were cultured in DMEM supplemented with these additional antibiotics: 6 μg/mL blasticidin and 100 μg/mL hygromycin.

C. Construction of Stable Cell Lines

For HEK293T-Based Stable Cell Lines:

Cells were trypsinized and split into 35 mm dishes one day before transfection. Confluent cells (60-70%) in 35 mm dishes were transfected with 1 μg of insect OR(X) plasmids in pME18s-bla and 1 μg of OR7-pME18s-puro using lipofectamine 2000. For Anopheles odorant receptors, two plasmids were co-transfected into HEK cells: the ligand-specific receptor (OR1, OR2, OR8, OR28) and the corresponding olfactory co-receptor: OR7. Cell lines containing TRPM8 were transfected with single plasmid.

After one day of incubation, the transfected cells were transferred into 10 cm dishes with DMEM containing antibiotics as follows:
0.5 μg/mL of blasticidin for OR1/OR7, OR2/OR7, OR8/OR7, OR10/OR7, OR28/OR7
0.2 μg/mL of puromycin for TRPM8.

The concentration of the above antibiotics was increased to 1.0 μg/mL when the population of cells became 30-40% confluent (typically on the seventh day of incubation). At or around the 10^{th} day of incubation, 1.0 μg/mL of puromycin was added into the plates for OR1, OR2, OR8, OR10, OR28.

After this point, cells were cultured by changing medium every two days until cell clumps appeared. The clumps were picked and transferred into 96 well plates with DMEM containing antibiotics: lug/mL of blasticidin and puromycin for OR1, OR2, OR8, OR10, OR28, and lug/mL for TRPM8. The cells were grown and transferred to 24 well plates, and ligand-evoked calcium increases were tested using the Hamamatsu FDSS6000 real-time fluorescence plate reader.

For Flip-In T-Rex based Stable Cell Lines:

Cells were trypsinized and split into 35 mm dishes 1 day before transfection. Confluent cells (60-70%) in 35 mm dishes were co-transfected with 1 μg of Or47a/Or83b plasmid in pcDNA5/FRT/TO/IRES plus the pOG44 plasmid or 1 μg of 5HT3 plasmid in pcDNA5/FRT/TO plus the pOG44 plasmid using lipofectamine 2000. Co-transfection of pOG44 and pcDNA5/FRT/TO/IRES or pcDNA5/FRT/TO allows expression of Flp recombinase and integration of the pcDNA5 plasmid into the genome via the FRT site. After one day of incubation, the transfected cells were transferred into 10 cm dishes with DMEM containing antibiotics: 20 μg/mL of hygromycin and 6 μg/mL of blasticidin. The concentration of Hygromycin was increased to 100 μg/mL when the population of cells became 30-40% confluent. The cells were cultured by changing medium every two days until cell clumps appeared. The clumps were picked and transferred into 96 well plates with DMEM containing 100 μg/mL of Hygromycin and 6 μg/mL of blasticidin. The cells were grown and transferred to 24 well plates, and ligand-evoked calcium increases were tested using the Hamamatsu FDSS6000 real time fluorescence plate reader.

D. Ca²⁺-Imaging of HEK Cells Using the Hamamatsu FDSS6000 Real Time Fluorescent Plate Reader

Stable cell lines were cultured in 10 cm dishes, and confluent cells (70-80%) were trypsinized. 10,000 cells/20 μL were transferred into each well of 384-well plates coated with poly-D-lysine, and cultured for 2 days. For Flip-In T-Rex cells (cell lines expressing Or47a/Or83b and 5HT3), 5 μL of tetracycline (5 μg/mL) were added 1 day before the assay. 20 μL of Ca-4 dye was loaded into each well for 1.5 hr at room temperature in the dark, and the dye-loaded plates were subjected to Ca²⁺-imaging using a Hamamatsu FDSS6000 real time fluorescence plate reader. Odorant solutions (9× final concentration) were prepared by dissolving 1M stock solutions into Hank's Buffered Salt Solution (HBSS), and 30 μL of odor solution was transferred to each well in 384-well plates. The last two columns of the plate (32 wells total) were used as control wells as follows:
negative control with HBSS buffer (16 wells)
positive control with ligands (16 wells).

Compounds (5 M stock in DMSO) were stored in 384-well plates, and last two columns (32 wells total) contained only DMSO. 200 nL of small compounds were transferred into the odorant plates before the assay using the Perkin-Elmer Janus workstation. The odorant plates were spun down to remove air bubbles, and placed on the FDSS6000 plate loader, and 5 μL of odorant solution was applied to the cell plate on the FDSS6000. Ca²⁺ increase was monitored with fluorescence at 525 nm by excitation at 485 nm.

E. Single Sensillum Recordings of Fly (Drosophila melanogaster) Olfactory Neurons

Female flies (Drosophila melanogaster) of the w¹¹¹⁸ genotype were immobilized by inserting single adult flies into the wide end of a 200 μL pipette tip, and mounting this on a microscope cover glass with a piece of wax. Antennal olfactory neurons of immobilized flies were subjected to extracellular recording using a Syntech 10x AC probe connected to a Syntech IDAC4 amplifier. The recording electrode was inserted into the ab5 sensillum, which contains two olfactory sensory neurons (OSNs), and the reference electrode was placed into the eye. Action potentials in the OSNs induced by odorant stimulation were recorded. 15 μL of odorant [3-methyl-thio-1-propanol; diluted (v/v) in paraffin oil (10⁻⁵ to 10⁻³ dilution)], was applied to a filter strip, and 10 μL of test compounds, DEET, or solvent (100% ethanol) was applied on a second filter strip. Both filter strips were placed into a Pasteur glass pipette connected to the Syntech CS55 stimulus device, and applied to the fly antenna for 1 sec. Corrected spike increases were computed by subtracting the average spontaneous activity in 1 sec before odorant stimulus from the average activity during the stimulus.

F. Fly (Drosophila melanogaster) Behavior Assays

Male flies (Drosophila melanogaster) of the w¹¹¹⁸ genotype were “wet-starved” for 24 hours in plastic culture vials lacking food but supplemented with wet cotton plugs on the bottom. After the 24 hour fasting period before the assay, flies were placed into a two-choice behavior chamber. This two-choice chamber consists of one 15 cm Petri dish with two round holes (2.2 cm diameter) that were punched along the midline at an equal distance (2.9 cm) from the rim. Two small plastic vials were connected to the dish and plugged with rigid cotton plugs perforated with 1 mL pipette tips. The bottom of each trap vial was humidified with the same cotton plugs used to plug the vial by soaking the plug in deionized water for a few seconds until it was fully wetted. Each trap contained a small filter paper strip with 10 μL of either water or pure odor (3-methyl-thio-1-propanol). Flies could enter one of the two traps through a plastic pipette tip (1 mL) inserted into top of each vial. 10 μL of RU-14, or solvent (100% ethanol) or DEET was placed on small circular filter paper, and introduced into the pipette tips at the entrance. A perforated 200 μL pipette tip was used to cover the filter paper to prevent direct contact of animals with compounds or DEET. All assays were conducted at 25° C. and 70-80% humidity with 40 starved male flies. After 24 hours, flies were counted and a response was computed by calculating the percent of flies in each trap. Significance was assessed using the Mann-Whitney U-test.

G. Mosquito (Aedes aegyptt) Behavioral Assays

Female Aedes aegypti Orlando mosquitoes (8-11 days old) were used in this assay. Larvae were hatched from dried egg papers and were reared in pans with deionized water supplemented with two tablets of fish food “TetraMin®” from Tetra every day. Pupae were transferred to cages and maintained before and after eclosion at 25° C. and 70-80% humidity with a 12 hr light: 12 hr dark photocycle. Adults were fed a 10% corn syrup solution and were free to mate with each other. One day prior to the assay, females were separated and placed into starvation cartridges (each containing 20 mosquitoes) without any food or water. The cartridges were placed at 25° C. and 70-80% humidity overnight (>l4hr).

A two-port olfactometer, adapted from Gouck (Gouck, H. K. Host preferences of various strains of Aedes aegypti and Aedes simpsoni as determined by an olfactometer. Bull. World Health Org. 47:680-683, 1972) was used to assess the repellent characteristic of small compounds by measuring mosquito host-seeking preference to human odor with a compound or its solvent. The two-port olfactometer consists of the large plastic box (main compartment 50×50×80 cm) and two trapping chambers and sock ports attached to the main compartment, and white cloth barrier was covered on the device to minimize visual distraction. 50 female adult mosquitoes, Aedes aegypti Orlando strain, (12-20 days after adult eclosion) were anaesthetized in a 4° C. room and sorted into separate starvation cartridge without any food or water one day prior to the assay. The cartridges were placed at 25° C.±0.5° C. and 78±5% humidity overnight (>14 h). All behavioral experiments were performed at the same condition. Ten minutes prior to the assay, mosquitoes were released and equilibrated in the main compartment. During this equilibration period, 100 μL of compound, DEET, solvent (100% ethanol), was pipetted onto a filter paper (2×5 cm), and allowed to air dry outside the apparatus for 9 min. The filter paper was hung perpendicular to the sock port opening. The odor bait (human or blank) consisting of a strip of sock made of 100% nylon and measuring 3×30 cm, was placed in the sock port. For “human” socks, socks were worn for 24 hours before the assay and cut into six pieces. Once mosquitoes were equilibrated, a sliding door between the trapping chambers and main compartment was opened to allow air and CO2 to flow downwind through the apparatus. 10% CO2 was applied through plastic tubing into the sock ports, producing a final concentration of 4% CO2 in the air flow in the trapping chambers. After 8 minutes, the number of mosquitoes in the trapping chambers (“attracted”) and main compartment (“not-attracted”) was counted. Data were expressed as % attracted/% activated. Significance was assessed using the analysis of variance, ANOVA. From a single whole socks, the first trial “solvent vs solvent” was carried out as a control for sock potency and the socks, which showed more than 60% attraction, were subjected to the assay, and then on the remaining two trials, “compound vs solvent” were performed. The placement of the compound was changed between trials to eliminate potential side bias. Significance was assessed using the Mann-Whitney U-test.

Experimental Results

In Vivo Screening Results

The amount of compound that reaches the insect antenna will be a portion of the applied dose, and the ratio of perceived dose to applied dose will be a function of the volatility of the compound being tested. For compounds of low volatility the ratio can be small.

A. Single Sensillum Recordings of Fly (Drosophila melanogaster) Olfactory Neurons

The effect of selected compounds on odor-evoked action potentials was measured from the ab5 antennal sensillum, which houses the olfactory neuron that expresses the Or47a/Or83b receptor.

The following compounds showed statistically significant suppression of 3-methyl-thio-1-propanol (3MTP)-evoked action potentials in the ab5 neuron:

RU-I1 When supplied at a 3.5 mg dose on a filter paper, this compound inhibits responses elicited by 5×10⁻⁴ to 10⁻⁵ dilutions of 3MTP.
When supplied at a 10 mg dose on a filter paper, this compound inhibits responses elicited by 10⁻³ to 10⁻⁵ dilutions of 3MTP.

RU-I2 When supplied at a 3.5 mg dose on a filter paper, this compound inhibits responses elicited by 10⁻⁴ to 10⁻⁵ dilutions of 3MTP.
When supplied at a 10 mg dose on a filter paper, this compound inhibits responses elicited by 10⁻³ to 10⁻⁵ dilutions of 3MTP.

RU-I5 When supplied at a 10 mg dose on a filter paper, this compound inhibits responses elicited by 10⁻³ to 5×10⁻⁵ dilutions of 3MTP.

B. Fly (Drosophila melanogaster) Behavior Assays

The effect of selected compounds on inhibition of attraction to 3 -methyl-thio-1-propanol (3MTP) was tested in a two-choice behavior assay. Both traps contained the same concentration of 3MTP, but the entrance to one trap was treated with a given compound.

RU-I4 showed significant suppression of 3MTP attraction when supplied at a 3.5 mg dose. DEET shows significant suppression in the range of 1 mg to 10 mg, indicating that RU-I4 has efficacy in the same concentration range as DEET.

C. Mosquito (Aedes aegyptt) Behavioral Assays

The effect of selected compounds on inhibition of attraction of female mosquitoes to human hand odor and CO₂ was measured in a two-port olfactometer assay. Compounds RU-I2 and RU-I5 showed significant inhibition of host-seeking behavior when supplied at a 3.5 mg dose.

### Data Tables

Table A (below) shows the Percent Inhibition Data for selected compounds applied at 4 μM across the receptor panel. The Percent Inhibition at 4 μM compound concentration is defined as follows:

A=>75% inhibition

B=51-74% inhibition

C=26% -50% inhibition,

D<25% inhibition (D defined as inactive)

ND=not determined

TABLE A
Percent Inhibition Data for Selected Compounds applied at 4 μM Across
Receptor Panel

| Compound | OR8 | OR10 | OR28 | OR47a | | | |
|---|---|---|---|---|---|---|---|
| ID | OR7 | OR7 | OR7 | OR83b | mOREG | TRPM8 | 5HT3 |
| RU-0000619 | C | D | B | B | B | D | C |
| RU-0001534 | C | D | A | B | D | D | C |
| RU-0006256 | B | B | A | B | D | C | C |
| RU-0015806 | D | C | A | B | D | C | B |
| RU-0018338 | D | C | B | B | C | C | C |
| RU-0020050 | A | D | A | B | C | C | C |
| RU-0026074 | ND | C | A | B | D | C | D |
| RU-0028727 | ND | B | A | A | B | C | B |
| RU-0029350 | C | B | A | B | C | C | D |
| RU-0029409 | C | C | A | B | D | C | D |
| RU-0030324 | C | C | A | B | B | C | C |
| RU-0032455 | C | D | A | B | C | D | C |
| RU-0032465 | B | D | A | B | C | C | D |
| RU-0032474 | D | D | B | B | D | D | D |
| RU-0034108 | D | C | C | C | D | D | D |
| RU-0036855 | C | D | A | C | C | C | D |
| RU-0036875 | B | C | A | B | B | D | B |
| RU-0036876 | B | D | A | B | C | D | C |
| RU-0036983 | B | C | A | B | C | C | C |
| RU-0037003 | C | D | A | D | C | D | C |
| RU-0037275 | B | D | A | B | C | D | C |
| RU-0037278 | C | C | A | B | C | D | C |
| RU-0037386 | B | C | A | B | C | D | C |
| RU-0037434 | B | C | A | B | C | D | C |
| RU-0038795 | D | D | A | B | C | D | B |
| RU-0039123 | C | C | A | B | C | D | C |
| RU-0045351 | D | D | A | C | C | D | D |
| RU-0045678 | C | A | A | C | C | D | C |
| RU-0045691 | D | C | A | C | C | D | D |
| RU-0045756 | D | D | B | C | C | D | D |
| RU-0045778 | D | D | B | C | C | D | C |
| RU-0047281 | A | A | A | A | C | B | A |
| RU-0047568 | C | A | A | C | C | C | C |
| RU-0047694 | B | B | A | C | D | D | C |
| RU-0047765 | D | B | A | C | D | C | D |
| RU-0047851 | D | A | C | C | C | D | D |
| RU-0047955 | D | A | A | C | D | D | D |
| RU-0048046 | D | B | A | C | D | D | D |
| RU-0048001 | C | A | A | B | C | C | D |
| RU-0048054 | D | B | D | C | D | D | D |
| RU-0048066 | D | B | A | C | D | D | D |
| RU-0048504 | D | B | A | C | D | D | D |
| RU-0048608 | D | B | A | C | D | D | D |
| RU-0050325 | C | D | A | C | D | D | D |
| RU-0050344 | C | C | A | C | C | D | D |
| RU-0050372 | D | C | A | C | D | D | D |
| RU-0050384 | D | D | A | C | D | D | D |
| RU-0050390 | D | D | A | C | D | D | D |
| RU-0050393 | C | B | A | C | C | D | C |
| RU-0050398 | D | D | A | C | D | D | D |
| RU-0050405 | C | C | A | B | D | D | D |
| RU-0051926 | D | C | A | C | D | D | B |
| RU-0053556 | C | B | A | C | C | C | D |
| RU-0055322 | D | B | A | C | C | D | D |
| RU-0055364 | C | A | A | C | C | D | D |
| RU-0058232 | C | A | A | C | C | C | B |
| RU-0058298 | D | A | A | B | C | C | C |
| RU-0058550 | D | C | A | C | C | D | D |
| RU-0058964 | C | A | A | B | C | C | B |
| RU-0071273 | C | B | A | C | C | D | C |
| RU-0071628 | C | D | A | B | C | D | C |
| RU-0071789 | D | A | A | C | C | D | C |
| RU-0071818 | D | B | A | C | B | D | D |
| RU-0071828 | D | C | A | C | C | D | C |
| RU-0072462 | D | C | A | C | D | D | D |
| RU-0075445 | D | D | A | D | D | D | D |
| RU-0075446 | D | A | A | D | D | D | C |
| RU-0075468 | D | C | A | D | A | D | D |
| RU-0075475 | D | A | A | D | D | D | D |
| RU-0001327 | C | B | A | D | C | D | C |
| RU-0001617 | D | B | B | B | D | D | C |
| RU-0023245 | ND | C | A | A | C | D | C |
| RU-0025771 | ND | C | A | A | C | C | C |
| RU-0026167 | C | C | B | B | C | C | D |
| RU-0026507 | D | D | A | C | D | D | C |
| RU-0028029 | B | C | B | A | D | C | C |
| RU-0031879 | C | D | A | B | C | C | D |
| RU-0035287 | C | D | A | B | C | D | C |
| RU-0036264 | C | D | B | D | D | C | D |
| RU-0036294 | B | B | A | B | C | C | D |
| RU-0043595 | C | D | A | B | B | D | B |
| RU-0045115 | D | D | A | C | C | D | D |
| RU-0046426 | C | C | A | C | B | D | C |
| RU-0047750 | D | A | A | C | D | D | C |
| RU-0049030 | C | C | A | C | D | D | D |
| RU-0051652 | D | C | B | D | D | D | C |
| RU-0052340 | D | B | A | C | C | D | D |
| RU-0052360 | B | A | A | C | C | C | D |
| RU-0069950 | C | A | A | B | B | D | B |
| RU-0072008 | D | A | A | C | C | D | D |
| RU-0072535 | D | D | A | C | C | D | D |
| RU-0074560 | D | D | B | C | D | D | D |
| RU-0076832 | D | C | A | D | C | D | C |
| RU-0077733 | D | C | B | C | D | D | D |
| RU-0079120 | D | A | A | B | D | D | C |

TABLE B
IC₅₀ Data for Selected Compounds Across Receptor Panels
IC₅₀ values defined as: F1 < 0.1 μM, F2 = between 0.1-1.0 μM,
F3 > 1 μM

| Compound | | | | | | | |
|---|---|---|---|---|---|---|---|
| ID | OR8 | OR10 | OR28 | OR47a | mOREG | TRPM8 | 5HT3 |
| RU-0000619 | | | F1 | F3 | F3 | | |
| RU-0001534 | | | F1 | F3 | | | |
| RU-0006256 | F3 | F3 | F1 | F3 | | | |
| RU-0015806 | | F3 | F1 | F1 | | | F3 |
| RU-0018338 | | | F1 | F1 | | | |
| RU-0020050 | F3 | | F2 | F1 | | | |
| RU-0026074 | | | | F3 | | | |
| RU-0028727 | | F3 | F1 | F1 | F3 | | F3 |
| RU-0029350 | F3 | F3 | F2 | F1 | | | |
| RU-0029409 | | | F1 | | | | |
| RU-0030324 | | | F1 | F1 | F3 | | |
| RU-0032455 | | | F1 | F3 | | | |
| RU-0032465 | F3 | | F1 | F3 | | | |
| RU-0032474 | | | F1 | F3 | | | |
| RU-0034108 | | | | F3 | | | |
| RU-0036855 | | | F1 | | | | |
| RU-0036875 | F3 | | F1 | F3 | F3 | | F3 |
| RU-0036876 | F3 | | F1 | F3 | | | |
| RU-0036983 | F3 | | F1 | F3 | | | |
| RU-0037003 | | | F1 | | | | |
| RU-0037275 | F3 | | F1 | F3 | | | |
| RU-0037278 | | | F1 | F3 | | | |
| RU-0037386 | F3 | | F1 | F3 | | | |
| RU-0037434 | F3 | | F1 | F3 | | | |
| RU-0038795 | | | F1 | F3 | | | F3 |
| RU-0039123 | | | F1 | F3 | | | |
| RU-0045351 | | | F1 | | | | |
| RU-0045678 | | F3 | F1 | | | | |
| RU-0045691 | | | F1 | | | | |
| RU-0045756 | | | F3 | | | | |
| RU-0045778 | | | F1 | | | | |
| RU-0047281 | F3 | | F1 | F3 | | F3 | F2 |
| RU-0047568 | | F1 | F1 | | | | |
| RU-0047694 | F2 | F1 | F1 | | | | |
| RU-0047765 | | F1 | F1 | F3 | | | |
| RU-0047851 | | F1 | F1 | | | | |
| RU-0047955 | | F1 | F1 | | | | |
| RU-0048001 | | F1 | F1 | F3 | | | |
| RU-0048046 | | F1 | F1 | | | | |
| RU-0048054 | | F1 | F1 | | | | |
| RU-0048066 | | F1 | F1 | | | | |
| RU-0048504 | | F1 | F1 | | | | |
| RU-0048608 | | F3 | F1 | | | | |
| RU-0050325 | | | F1 | F3 | | | |
| RU-0050344 | | | F1 | | | | |
| RU-0050372 | | | F1 | | | | |
| RU-0050384 | | | F2 | | | | |
| RU-0050390 | | | F1 | | | | |
| RU-0050393 | | F3 | F1 | | | | |
| RU-0050398 | | | F2 | | | | |
| RU-0050405 | | F3 | F1 | F3 | | | |
| RU-0051926 | | | F3 | | | | F2 |
| RU-0053556 | | F3 | F2 | | | | |
| RU-0055322 | | F1 | F1 | | | | |
| RU-0055364 | | F2 | F1 | | | | |
| RU-0058232 | | F1 | F1 | | | | F3 |
| RU-0058298 | | F1 | F1 | F3 | | | |
| RU-0058550 | | | F1 | | | | |
| RU-0058964 | | F1 | F1 | F3 | | | F3 |
| RU-0071273 | | F1 | F1 | F3 | F2 | | |
| RU-0071628 | | | F1 | F3 | | | |
| RU-0071789 | | F1 | F1 | | | | |
| RU-0071818 | | F3 | F1 | | F3 | | |
| RU-0071828 | | F3 | F1 | | | | |
| RU-0072462 | | | F1 | | | | |
| RU-0075445 | | | F1 | | | | |
| RU-0075446 | | F1 | F1 | | | | |
| RU-0075468 | | F1 | F1 | | F1 | | |
| RU-0075475 | | F1 | F3 | | | | |
| RU-0001327 | | F3 | F3 | | | | |
| RU-0001617 | | F1 | F3 | F3 | | | |
| RU-0023245 | | F3 | F3 | F3 | | | |
| RU-0025771 | | | | F3 | | | |
| RU-0026167 | | | F1 | F3 | | | |
| RU-0026507 | | | F3 | | | | |
| RU-0028029 | F3 | | F1 | F3 | | | |
| RU-0031879 | | | F1 | F3 | | | |
| RU-0035287 | | | F1 | F1 | | | |
| RU-0036264 | | | F2 | | | | |
| RU-0036294 | F3 | F3 | F2 | | | | |
| RU-0043595 | | | F1 | F3 | F3 | | F3 |
| RU-0045115 | | | F1 | | | | |
| RU-0046426 | | | F3 | | F3 | | |
| RU-0047750 | | F1 | F3 | | | | |
| RU-0049030 | | | F1 | | | | |
| RU-0051652 | | | F3 | | | | |
| RU-0052340 | | F1 | F1 | | | | |
| RU-0052360 | F3 | F3 | F2 | | | | |
| RU-0069950 | | F1 | F1 | F3 | F3 | | F3 |
| RU-0072008 | | F1 | F1 | | | | |
| RU-0072535 | | | F1 | | | | |
| RU-0074560 | | | F3 | | | | |
| RU-0076832 | | | F1 | | | | |
| RU-0077733 | | | F1 | | | | |
| RU-0079120 | | F1 | F1 | F1 | | | |

TABLE C
IC₅₀ Values of Compounds Across Receptor Panel

| | Receptors: | | | | | | | | |
|---|---|---|---|---|---|---|---|---|---|
| | OR1 | OR2 | OR8 | OR10 | OR28 | OR47a | | | |
| | OR7 | OR7 | OR7 | OR7 | OR7 | OR83b | P2YR | TRPM8 | 5HT3 |
| | Ligand: | | | | | | | | |
| | 4-methyl | 2-methyl | | | | pentyl | | | |
| | phenol | phenol | 1-octen-3-ol | indole | acetophenone | acetate | ATP | menthol | serotonin |
| Compound | Ligand Screening Concentration (μM) | | | | | | | | |
| ID | 30 μM | 30 μM | 30 μM | 30 μM | 300 μM | 30 μM | 1 μM | 300 μM | 10 μM |
| RU-T1 | ++++ | ++++ | IA | P | IA | P | IA | IA | IA |
| RU-T2 | +++ | +++ | ++++ | +++ | +++ | P | IA | IA | +++ |
| RU-T3 | P | +++ | +++ | +++ | +++ | +++ | IA | P | ++ |
| RU-T4 | +++ | ++ | +++ | ++ | P | P | IA | IA | ++ |
| RU-T5 | ++++ | ++++ | ++++ | +++ | P | P | IA | IA | P |
| RU-T6 | +++ | P | ++++ | P | ++ | P | IA | IA | IA |
| RU-T7 | ++++ | ++++ | ++++ | P | P | P | IA | IA | P |
| RU-T8 | ++++ | ++++ | +++ | P | P | P | IA | IA | P |
| RU-T9 | ++++ | P | P | P | + | P | IA | IA | + |
| RU-I1 | ++++ | ++++ | ++ | +++ | ++ | P | IA | IA | ++ |
| RU-I2 | ++++ | ++++ | +++ | +++ | +++ | ++++ | IA | P | +++ |
| RU-I3 | ++++ | +++ | ++++ | +++ | ++++ | ++++ | IA | P | +++ |
| RU-I4 | ++++ | +++ | P | P | +++ | P | IA | IA | P |
| RU-I5 | ++++ | ++++ | +++ | P | +++ | ++ | IA | P | +++ |
| DEET | IA | P | + | + | + | + | IA | P | + |

IC₅₀ values defined as:
++++ <2 μM
+++ Between 2-20 μM
++ Between 20-50 μM
+ >50 μM
Partial activity: highest concentration assayed (>20 μM for T6, T7, I1, I2; >70 μM for T4, I5; >220 μM for T1, T2, T3, T5, T8, T9, I3, I4; >740 μM for DEET) did not reach 50%
P inhibition
IA Inactive: compound did not show inhibition over 5-log concentration range

1. A method for interfering with the ability of an insect to detect odors, the method comprising exposing said insect to an olfactory-disrupting concentration of a compound chosen from the following five genera: 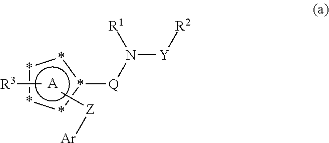
wherein
A is a five-membered, aromatic heterocycle in which one, two or three of the vertices marked by asterisks are heteroatoms chosen from nitrogen, oxygen and sulfur, and the remaining vertices are carbon;
Ar is optionally substituted aryl or heteroaryl;
Z is chosen from a direct bond, —CH₂—, —O—, —CH₂CH₂—, —CH₂O— and —OCH₂—;
Q is chosen from —(CH₂)ₘ—, wherein one or more —CH₂— may be replaced by —C═O—, —SO₂—, —NH—, —S—, —Ar^{a}—, —CHOH— or —O—;
Ar^{a} is optionally substituted aryl or heteroaryl;
Y is chosen from —(CH₂)ₘ—, wherein one or more —CH₂— may be replaced by —C═O—, —SO₂—, —NH—, —S—, —CHOH— or —O—;
R¹ and R² are independently chosen from H, (C₁-C₁₀)hydrocarbon, (C₁-C₁₀)oxaalkyl, aryl, substituted aryl, heterocyclyl, substituted heterocyclyl, —(CH₂)ₘR¹⁰ and —(CH₂)—CHR¹¹—CH₂—O—CH₂R¹⁰;
or taken together R¹ and R² form a 4-7 membered saturated monocycle or a 9-10 membered bicycle in which the ring formed by R¹—N—Y—R² is saturated, said monocycle or bicycle optionally substituted with one or two substituents chosen independently from halogen, (C₁-C₁₀)hydrocarbon, —C═O—, (C₁-C₁₀)oxaalkyl and —C(═O)R¹⁴;
R³ represents one or two residues chosen from hydrogen, (C₁-C₆)hydrocarbon, —NR¹²R¹³ , phenyl, and phenyl substituted with one or two substituents chosen from halogen, (C₁-C₄) alkyl, halo(C₁-C₄)alkyl, (C₁-C₄)alkoxy, and halo(C₁-C₄)alkoxy;
R¹⁰ is chosen from —COO(C₁-C₄)alkyl and monocyclic heterocycle;
R¹¹ is chosen from H, (C₁-C₁₀)hydrocarbon and —OH;
R¹² is chosen from H and (C₁-C₁₀)hydrocarbon;
R¹³ is chosen from H, (C₁-C₁₀)hydrocarbon, or R¹² and R¹³, taken together with the nitrogen to which they are attached, form a five- or six-membered ring optionally substituted with one or two substituents chosen from halogen, (C₁-C₈)hydrocarbon, and halo(C₁-C₄)alkyl;
R¹⁴ is chosen from H, (C₁-C₆)alkyl, (C₁-C₆)alkoxy and phenyl optionally substituted with one or two substituents chosen from halogen, (C₁-C₄)alkyl, halo(C₁-C₄)alkyl, (C₁-C₄)alkoxy, and halo(C₁-C₄)alkoxy; and
m is 1 to 5; or 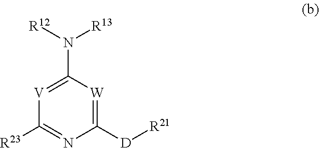
wherein V and W are independently chosen from N and CR¹²;
D is chosen from S, O and NR²⁴;
R²¹ is chosen from hydrogen, (C₁-C₁₀)hydrocarbon, —(CH₂)ₘCN, —(CH₂)ₘ—Ar^{a}, and —N═CH—Ar^{a};
R²³ is chosen from hydrogen, halogen, (C₁-C₁₀)hydrocarbon, halo(C₁-C₆)alkyl, (C₁-C₁₀)hydrocarbon-O— and —NR¹²R¹³;
R²⁴ is chosen from H, (C₁-C₆)hydrocarbon and —CN; or 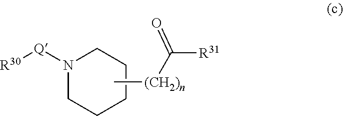
wherein
Q′ is chosen from —(CH₂)ₙC═O— and —(CH₂)ₙSO₂—, wherein the carbonyl or sulfonyl is the point of attachment to nitrogen;
n is 0, 1, 2, 3, 4 or 5 in each instance;
R³⁰ is chosen from —Ar^{a}, —O—Ar^{a}, —S—Ar^{a}, and (C₁-C₁₀)hydrocarbon; and
R³¹ is chosen from —Ar, —NR¹²Ar, —NR¹²CH₂Ar, and 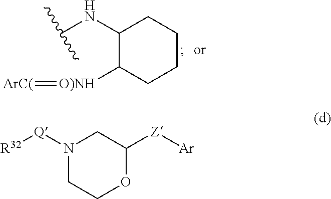
wherein
Z′ is chosen from , —CH₂—, —CH₂CH₂—, —CH₂O—, —CH₂OCH₂—, —CH₂CH₂CH₂— and —CH₂CH₂O—; and
R³² is chosen from —Ar^{a}, —OAr^{a}, —SAr^{a}, and —NHCOOCH₂Ar^{a}; or 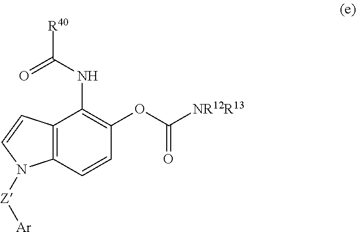
wherein
R⁴° is chosen from (C₁-C₁₀)hydrocarbon and an oxygenated (C₁-C₁₀)hydrocarbon.

2. (canceled)

3. A method according to claim 1 wherein said compound is of formula:

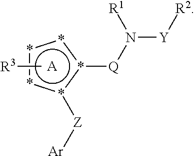

4. A method according to claim 3 wherein A is chosen from oxazole, thiazole, isothiazole, oxadiazole, thiadiazole, isoxazole, imidazole, pyrazole and triazole.

5. (canceled)

6. A method according to claim 1 wherein said compound is of formula:

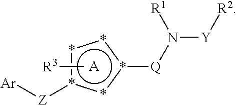

7-8. (canceled)

9. A method according to claim 1 wherein said compound is of formula 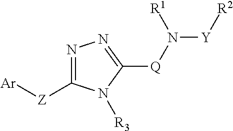
wherein Q is chosen from —S(CH₂)ₘC(═O)— and —S(CH₂)Ar^{a}C(═O)—;
R³ is optionally substituted phenyl; and
Ar is optionally substituted phenyl.

10. (canceled)

11. A method according to claim 1 wherein said compound is of formula: 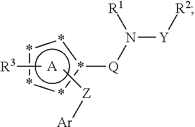
and
Ar is optionally substituted phenyl.

12. (canceled)

13. A method according to claim 1 wherein said compound is of formula: 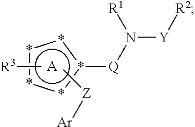
and
at least one of -Q- and —YR² contains oxygen.

14-15. (canceled)

16. A method according to claim 1 wherein said compound is of formula: 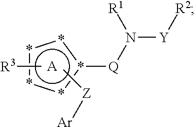
Y is —C(═O)—; and
R² is chosen from (C₁-C₆) alkyl, optionally substituted phenyl and furanyl.

17. A method according to claim 1 wherein said compound is of formula: 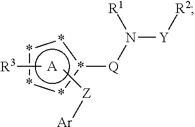
and
Q is CH₂.

18-20. (canceled)

21. A method according to claim 1 of formula

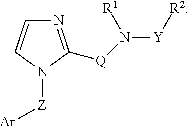

22. A method according to claim 21, wherein:
Z is CH₂;
Ar is phenyl optionally substituted with halogen;
Q is CH₂ or C═O;
Y is CH₂ or C═O;
R¹ is (C₁-C₁₀)hydrocarbon; and
R² is (C₁-C₁₀)hydrocarbon or phenyl optionally substituted with halogen.

23. A method according to claim 1 wherein said compound is of formula: 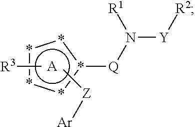
and
Z is selected from a direct bond and CH₂.

24. A method according to claim 1 wherein said compound is of formula: 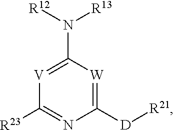
and both V and W are N.

25-31. (canceled)

32. A method according to claim 24 wherein
D is NR²⁴ or S;
R²⁴ is H or CH₃;
R²¹ is H or (C₁-C₁₀)hydrocarbon; and
R¹² and R¹³ are each independently selected from H and (C₁-C₁₀)hydrocarbon.

33. A method according to claim 1 wherein said compound is of formula: 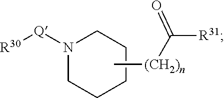
and
Q′ is —(CH₂)ₙ C═O— and n is zero to 2.

34-38. (canceled)

39. A method according to claim 1 wherein said compound is of formula: 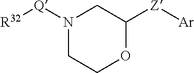
wherein
Z′ is chosen from, —CH₂—, —CH₂CH₂—, —CH₂O—, —CH₂OCH₂—, —CH₂CH₂CH₂— and —CH₂CH₂O—; and
R³² is chosen from —Ar^{a}, —OAr^{a}, —SAr^{a}, and —NHCOOCH₂Ar^{a}; and
—Ar^{a} is chosen from optionally substituted phenyl and optionally substituted pyridine.

40-42. (canceled)

43. A method according to claim 39 wherein the substituents are selected from —N[(C₁-C₆)hydrocarbon]₂, halogen, cyano and methoxy.

44-46. (canceled)

47. A method according to claim 1 wherein said compound is of formula: 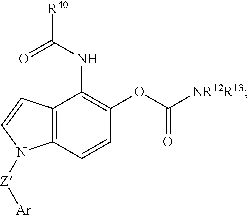
Z′ is —CH₂—.

48-51. (canceled)

52. A method for interfering with the ability of an insect to detect odors, the method comprising exposing said insect to an olfactory-disrupting concentration of a compound chosen from the compounds shown in the compound tables of the application.

53. An insect repellent composition suitable for topical application comprising a topically acceptable carrier and a compound described in claim 1; or
a topically acceptable carrier and a compound chosen from the compounds shown in the compound tables of the application.

54. (canceled)
